(19) United States
(12) Patent Application Publication
(10) Pub. No.: US 2022/0378780 A1
(43) Pub. Date: Dec. 1, 2022

(54) PHARMACEUTICAL COMPOSITIONS COMPRISING POH DERIVATIVES

(71) Applicant: NeOnc Technologies, Inc., Los Angeles, CA (US)

(72) Inventors: Thomas CHEN, La Canada, CA (US); Axel SCHÖNTHAL, Los Angeles, CA (US)

(21) Appl. No.: 17/762,878

(22) PCT Filed: Sep. 23, 2020
(86) PCT No.: PCT/US20/52190

§ 371 Date: Mar. 23, 2022

Related U.S. Application Data
(60) Provisional application No. 62/904,102, filed on Sep. 23, 2019.

(51) Int. Cl. A61K 31/495; A61K 31/415; A61K 31/4015; A61K 47/54; A61P 35/02

(52) U.S. Cl. CPC A61K 31/495; A61K 31/415; A61K 31/4015; A61K 47/54; A61P 35/02

(57) ABSTRACT

The present invention provides for a derivative of monoterpene or sesquiterpene, such as a perillyl alcohol derivative. For example, the perillyl alcohol derivative may be a perillyl alcohol carbamate. The perillyl alcohol derivative may be perillyl alcohol conjugated with a therapeutic agent such as a chemotherapeutic agent. The present invention also provides for a method of treating a disease such as a primary cutaneous lymphoma which may be a cutaneous T cell lymphoma (CTCL). The CTCL may be mycosis fungoides, primary cutaneous anaplastic large cell lymphoma (ALCL), or Sezary syndrome. A patient may be administered a therapeutically effective amount of a derivative of monoterpene (or sesquiterpene).

### FIELD OF THE INVENTION

The present invention relates to POH derivatives. The present invention further relates to methods of using POH derivatives such as POH carbamates to treat cancer.

### BACKGROUND OF THE INVENTION

Primary cutaneous lymphomas are a heterogenous group of extra-nodal non-Hodgkin lymphomas. In contrast to nodal non-Hodgkin lymphomas, most of which are B-cell derived, approximately 75% of primary cutaneous lymphomas are T-cell derived.¹ Cutaneous T-cell lymphomas (CTCL) are rare and they are characterized by the presence of malignant T-lymphocytes in the skin.^{2,3} They represent 3.9% of all non-Hodgkin lymphomas with an annual incidence of 6.4 to 9.6 cases per million people in the United States.⁴⁻⁶ Mycosis fungoides (MF) is the most common CTCL, whereas Sézary syndrome (SS) is much rarer. They account for 2-3% of all lymphomas⁷ and comprise approximately 53% of all cutaneous lymphomas.⁴ MF has an annual incidence of 5.6 per million persons representing 50% of all CTCL⁸, whereas SS has an annual incidence of 0.1-0.3 per million persons and represents 2.5% of all CTCL.⁹

Clinical symptoms of CTCL vary by subtype. In MF, a primarily cutaneous variant, symptoms remain mostly localized to the skin and include variably affected flat patches, thin plaques or tumors. In comparison, SS, a variant with a leukemic component, presents as a more aggressive phenotype, in which the skin is diffusely affected and there is greater involvement of the systemic circulation.¹⁰ In fact, the presence of >1,000 Sézary cells/mm³ in the circulation represents a key diagnostic criterion for SS. In both diseases, skin biopsies can reveal the characteristic Sézary cells (T cells with cerebriform nucleus) that are infiltrating the epidermis. While SS can arise as a progression of pre-existing MF, it more typically arises de novo and is generally considered a separate disease, rather than a leukemic progression of MF.^{11,12}

The etiology of MF and SS is still unknown. It is thought to include chronic antigenic stimulation through viral or bacterial exposure, environmental exposures, and altered microRNA (miRNA) expression.² A recent case series examined a subset of hypertensive MF patients using hydrochlorothiazide, speculating that this diuretic may be associated with antigen-driven T-cell lymphoproliferation and could serve as a trigger for MF. In addition, individual genetic features have also been implicated in the development of CTCL.¹ Furthermore, a variety of genetic aberrations have been identified in MF, such as mutations in the tumor suppressor p53 gene and loss of other tumor suppressor genes, such as CDKN2A and CDKN2B. As well, MF can have chromosomal gains and losses, and the Janus kinase (JAK) signal transducer and activator of transcription (STAT) pathways can be deregulated in MF and in CTCLs in general.^{1,2,13}

Treatment strategies range from an expectant policy in early-stage disease to hematopoietic stem cell transplantation, going through retinoids, immunotherapy, and extracorporeal photochemotherapy, among others.³ The National Comprehensive Cancer Network (NCCN) guidelines outline classic treatments for MF/SS as determined by stage of the disease, estimated skin tumor burden, presence of unfavorable prognostic factors, age and other comorbidities, such as cardiovascular disease, dyslipidemia, low thyroid function, etc., that can impact quality of life.¹⁴ Although there are several therapies recognized by the NCCN for the treatment of MF/SS, there is a paucity of effective therapies providing durable responses. Targeted therapies have variable response rates ranging from 30% to 67%, with complete responses no higher than 41%¹⁵ because none of these approaches are curative and patients frequently have relapses necessitating ongoing treatments.¹⁴ Even with extensive treatment, the prognosis of these diseases at their advanced stages remains poor. MF has a 27% 5-year survival in advanced disease, which in SS decreases to a 15% 5-year survival.⁷

Malignant gliomas, the most common form of central nervous system (CNS) cancers, is currently considered essentially incurable. Among the various malignant gliomas, anaplastic astrocytomas (Grade III) and glioblastoma multiforme (GBM; Grade IV) have an especially poor prognosis due to their aggressive growth and resistance to currently available therapies. The present standard of care for malignant gliomas consists of surgery, ionizing radiation, and chemotherapy. Despite recent advances in medicine, the past 50 years have not seen any significant improvement in prognosis for malignant gliomas. Wen et al. Malignant gliomas in adults. New England J Med. 359: 492-507, 2008. Stupp et al. Radiotherapy plus concomitant and adjuvant temozolomide for glioblastoma. New England J Med. 352: 987-996, 2005.

The poor response of tumors, including malignant gliomas, to various types of chemotherapeutic agents are often due to intrinsic drug resistance. Additionally, acquired resistance of initially well-responding tumors and unwanted side effects are other problems that frequently thwart long-term treatment using chemotherapeutic agents. Hence, various analogues of chemotherapeutic agents have been prepared in an effort to overcome these problems. The analogues include novel therapeutic agents which are hybrid molecules of at least two existing therapeutic agents. For example, cisplatin has been conjugated with Pt-(II) complexes with cytotoxic codrugs, or conjugated with bioactive shuttle components such as porphyrins, bile acids, hormones, or modulators that expedite the transmembrane transport or the drug accumulation within the cell. (6-Aminomethylnicotinate) dichloridoplatinum(II) complexes esterified with terpene alcohols were tested on a panel of human tumor cell lines. The terpenyl moieties in these complexes appeared to fulfill a transmembrane shuttle function and increased the rate and extent of the uptake of these conjugates into various tumor cell lines. Schobert et al. Monoterpenes as Drug Shuttles: Cytotoxic (6-minomethylnicotinate) dichloridoplatinum(II) Complexes with Potential To Overcome Cisplatin Resistance. J. Med. Chem. 2007, 50, 1288-1293.

Perillyl alcohol (POH), a naturally occurring monoterpene, has been suggested to be an effective agent against a variety of cancers, including CNS cancer, breast cancer, pancreatic cancer, lung cancer, melanomas and colon cancer. Gould, M. Cancer chemoprevention and therapy by monoterpenes. Environ Health Perspect. 1997 June; 105 (Suppl 4): 977-979. Hybrid molecules containing both perillyl alcohol and retinoids were prepared to increase apoptosis-inducing activity. Das et al. Design and synthesis of potential new apoptosis agents: hybrid compounds containing perillyl alcohol and new constrained retinoids. Tetrahedron Letters 2010, 51, 1462-1466.

There is still a need to prepare perillyl alcohol derivatives including perillyl alcohol conjugated with other therapeutic agents, and use this material in the treatment of cancers such as malignant gliomas, as well as other brain disorders such as Parkinson's and Alzheimer's disease. Perillyl alcohol derivatives may be administered alone or in combination with other treatment methods including radiation, standard chemotherapy, and surgery. The administration can also be through various routes including intranasal, oral, oral-tracheal for pulmonary delivery, and transdermal.

### SUMMARY OF THE INVENTION

The present disclosure provides for a pharmaceutical composition comprising a perillyl alcohol carbamate. The perillyl alcohol carbamate may be perillyl alcohol conjugated with a therapeutic agent, such as a chemotherapeutic agent. The chemotherapeutic agents that may be used in the present invention include a DNA alkylating agent, a topoisomerase inhibitor, an endoplasmic reticulum stress inducing agent, a platinum compound, an antimetabolite, an enzyme inhibitor, and a receptor antagonist. In certain embodiments, the therapeutic agents are dimethyl celocoxib (DMC), temozolomide (TMZ) or rolipram. The perillyl alcohol carbamates may be 4-(Bis-N,N′-4-isopropenyl cyclohex-1-enylmethyloxy carbonyl [5-(2,5-dimethyl phenyl)-3-trifluoromethyl pyrazol-1-yl] benzenesulfonamide, 4-(3-cyclopentyloxy-4-methoxy phenyl)-2-oxo-pyrrolidine-1-carboxylic acid 4-isopropenyl cyclohex-1-enylmethyl ester, and 3-methyl 4-oxo-3,4-dihydroimidazo[5,1-d][1,2,3,5]tetrazine-8-carbonyl)-carbamic acid-4-isopropenyl cyclohex-1-enylmethyl ester.

The pharmaceutical compositions of the present disclosure may be administered before, during or after radiation. The pharmaceutical compositions may be administered before, during or after the administration of a chemotherapeutic agent. The routes of administration of the pharmaceutical compositions include inhalation, intranasal, oral, intravenous, subcutaneous or intramuscular administration.

The disclosure further provides for a method for treating a disease in a mammal, comprising delivering to the mammal a therapeutically effective amount of a perillyl alcohol carbamate. The method may further comprise the step of treating the mammal with radiation, and/or further comprise the step of delivering to the mammal a chemotherapeutic agent.

The diseases treated may be cancer.

The diseases treated may be a tumor of the nervous system, such as a glioblastoma.

The routes of administration of the perillyl alcohol carbamate include inhalation, intranasal, oral, intravenous, subcutaneous or intramuscular administration.

The present disclosure provides for a method for treating a primary cutaneous lymphoma mycosis fungoides in a mammal, the method comprising administering to the mammal a therapeutically effective amount of a perillyl alcohol carbamate.

The primary cutaneous lymphoma may be a cutaneous T cell lymphoma (CTCL). The cutaneous T cell lymphoma (CTCL) may be mycosis fungoides, primary cutaneous anaplastic large cell lymphoma (ALCL), or Sezary syndrome. In one embodiment, the cutaneous T cell lymphoma (CTCL) is mycosis fungoides.

The perillyl alcohol carbamate may be perillyl alcohol conjugated with a therapeutic agent. The therapeutic agent may be a chemotherapeutic agent.

The chemotherapeutic agent may be a DNA alkylating agent, a topoisomerase inhibitor, an endoplasmic reticulum stress inducing agent, a platinum compound, an antimetabolite, an enzyme inhibitor, or a receptor antagonist.

The therapeutic agent may be dimethyl celecoxib (DMC), temozolomide (TMZ) or rolipram.

The perillyl alcohol carbamate may be (a) 4-(bis-N,N′-4-isopropenyl cyclohex-1-enylmethyloxy carbonyl [5-(2,5-dimethyl phenyl)-3-trifluoromethyl pyrazol-1-yl] benzenesulfonamide; (b) 4-(3-cyclopentyloxy-4-methoxy phenyl)-2-oxo-pyrrolidine-1-carboxylic acid 4-isopropenyl cyclohex-1-enylmethyl ester; or (c) 3-methyl 4-oxo-3,4-dihydroimidazo[5,1-d][1,2,3,5]tetrazine-8-carbonyl)-carbamic acid-4-isopropenyl cyclohex-1-enylmethyl ester.

The present method may further comprise treating the mammal with radiation.

The present method may further comprise administering to the mammal a chemotherapeutic agent.

The mammal may be a human.

The perillyl alcohol carbamate may be administered by inhalation, intranasally, orally, intravenously, subcutaneously or intramuscularly.

The present invention also provides for a process for making a POH carbamate, comprising the step of reacting a first reactant of perillyl chloroformate with a second reactant, which may be dimethyl celocoxib (DMC), temozolomide (TMZ) or rolipram. When the second reactant is dimethyl celocoxib, the reaction may be carried out in the presence of acetone and a catalyst of potassium carbonate. When the second reactant is rolipram, the reaction may be carried out in the presence of tetrahydrofuran and a catalyst of n-butyl lithium. The perillyl chloroformate may also be prepared by reacting perillyl alcohol with phosgene.

### BRIEF DESCRIPTION OF THE DRAWINGS

FIG. 1 shows the results of the MTT cytotoxicity assays demonstrating the efficacy of dimethyl celecoxib (DMC) in killing U87, A172 and U251 human glioma cells.

FIG. 2 shows the results of the MTT cytotoxicity assays demonstrating the efficacy of the POH-DMC conjugate in killing U87, A172 and U251 human glioma cells according to the present invention.

FIG. 3 shows the results of the MTT cytotoxicity assays demonstrating the efficacy of temozolomide (TMZ) in killing U87, A172 and U251 human glioma cells.

FIG. 4 shows the results of the MTT cytotoxicity assays demonstrating the efficacy of the POH-TMZ conjugate in killing U87, A172, and U251 human glioma cells according to the present invention.

FIG. 5 shows the results of the MTT cytotoxicity assays demonstrating the efficacy of the POH-Rolipram conjugate and Rolipram in killing A172 human glioma cells.

FIG. 6 shows the results of the MTT cytotoxicity assays demonstrating the efficacy of the POH-Rolipram conjugate and Rolipram in killing U87 human glioma cells.

FIG. 7 shows the results of the MTT cytotoxicity assays demonstrating the efficacy of the POH-Rolipram conjugate and Rolipram in killing U251 human glioma cells.

FIG. 8 shows the results of the MTT cytotoxicity assays demonstrating the efficacy of the POH-Rolipram conjugate and Rolipram in killing L229 human glioma cells.

FIGS. 9A-9B show the inhibition of tumor growth by butyryl-POH in mouse models.

FIG. 9A shows the images of subcutaneous U-87 gliomas in nude mice treated with butyryl-POH, purified (S)-perillyl alcohol having a purity greater than 98.5% (“Purified POH”), POH purchased from Sigma chemicals (“Sigma”), or phosphate buffered saline (“PBS”; negative control). FIG. 9B shows average tumor growth over time (total time period of 60 days).

FIG. 10 shows the results of a Colony forming Assay (CFA) demonstrating the cytotoxic effect of TMZ and TMZ-POH on TMZ sensitive (U251) and TMZ resistant (U251TR) U251 cells.

FIG. 11 shows the results of a Colony forming Assay (CFA) demonstrating the cytotoxic effect of POH on TMZ sensitive (U251) and TMZ resistant (U251TR) U251 cells.

FIG. 12 shows the results of the MTT cytotoxicity assays demonstrating the efficacy of the POH-TMZ conjugate in killing U251 cells, U251TR cells, and normal astrocytes.

FIG. 13 shows the results of the MTT cytotoxicity assays demonstrating the efficacy of the POH-TMZ conjugate in killing normal astrocytes, brain endothelial cells (BEC; confluent and subconfluent), and tumor brain endothelial cells (TuBEC).

FIG. 14 shows the results of the MTT cytotoxicity assays demonstrating the efficacy of TMZ and the POH-TMZ conjugate in killing USC-04 glioma cancer stem cells.

FIG. 15 shows the results of the MTT cytotoxicity assays demonstrating the efficacy of POH in killing USC-04 glioma cancer stem cells.

FIG. 16 shows the results of the MTT cytotoxicity assays demonstrating the efficacy of TMZ and the POH-TMZ conjugate in killing USC-02 glioma cancer stem cells.

FIG. 17 shows the results of the MTT cytotoxicity assays demonstrating the efficacy of POH in killing USC-02 glioma cancer stem cells.

FIG. 18 shows a western blot demonstrating that TMZ-POH induces ER stress (ERS) in TMZ sensitive (“U251-TMZs”) and resistant (“U251-TMZr”) U251 glioma cells.

FIG. 19 shows the results of the MTT cytotoxicity assays demonstrating the efficacy of the POH-TMZ conjugate and the triple conjugate of temozolomide (TMZ), perillyl alcohol (POH), and linoleic acid in killing HuT 78 mycosis fungoides cells in vitro. HuT 78 cells were treated with (i) temozolomide (50, 100, 250 μM), (ii) POH-TMZ (“NEO212”, 25, 50, 100 μM), (iii) NEO412 which is the triple conjugate of TMZ, POH, and linoleic acid (25, 50, 100, 250 μM), or (iv) vehicle alone. Seventy-two hours after the addition of drugs or vehicle, cell viability was determined by standard MTT (methylthiazoletetrazolium) assay.

FIGS. 20A-20C. NEO212 reduces cell viability. Cells were exposed to increasing concentrations of NEO212, or vehicle only, or remained untreated. At different time points thereafter, standard MTT cell viability assay was performed. (FIG. 20A) HUT-78 cells. (FIG. 20B) HUT-102 cells. (FIG. 20C) Myla cells. In all cases, viability of untreated cells was set to 100%. Vehicle-treated cells did not show differences to untreated cells.

FIGS. 21A-21C. NEO212 reduces cell proliferation. Cells were exposed to increasing concentrations of NEO212 or remained untreated. At different time points thereafter, viable cells were counted via Trypan blue exclusion. (FIG. 21A) HUT-78 cells. (FIG. 21B) HUT-102 cells. (FIG. 21C) Myla cells. Asterisks: *: p<0.05; **: p<0.01 (as compared to untreated cells).

FIGS. 22A-22C. NEO212 is more cytotoxic than its individual constituents. Cells were exposed to increasing concentrations of NEO212, TMZ, POH, or TMZ in combination with POH (TMZ+POH). After 72 hours, standard MTT cell viability assay was performed. (FIG. 22A) HUT-78 cells. (FIG. 22B) HUT-102 cells. (FIG. 22C) Myla cells. In all cases, viability of untreated cells was set to 100%. Vehicle-treated cells did not show differences to untreated cells. Shown is average (n=4)±standard deviation.

FIG. 23. Differential expression of MGMT protein in MF and SS cells. Total cell lysates were subjected to Western blot analysis for MGMT expression. For comparison purposes, lysates from two glioblastoma cell lines, U251 (MGMT-negative) and T98G (MGMT-positive) were included. Actin was used as a loading control.

FIG. 24. NEO212 triggers cell death more potently than TMZ. HUT-78 cells were exposed to increasing concentrations of NEO212 or TMZ for 72 hours. As a positive control, some cells received staurosporine (STSP) for 24 hours. Thereafter, cells were stained with 7-AAD and subjected to FACS. Note that the lowest concentration of NEO212 (1 μM) was more potent than the highest concentration of TMZ (300 μM).

FIGS. 25A-25D. NEO212 induces protein markers of apoptosis. HUT-78 cells (FIG. 25A), HUT-102 cells (FIG. 25B), and MyLa cells (FIG. 25C) were treated with increasing concentrations of NEO212 or vehicle (vh.). A and B were treated for 72 hours, and C for 96 hours. (FIG. 25D) MyLa cells received repeated treatments of NEO212: 25 and 50 μM NEO212 were added once per day for 5 consecutive days (5×), whereas 75 μM NEO212 was added once per day for 3 consecutive days (3×). Vehicle (vh.) was added once per day for 5 consecutive days (5×). Cells were harvested 24 hours after the final addition of NEO212 (or vehicle). In all cases, total cell lysates were prepared and subjected to Western blot analysis with specific antibodies to markers of cell death, including activated (i.e., cleaved, cl.) caspases, and PARP-1. For the latter, arrows point to its full-length (f.l.) and cleaved (cl.) form. Actin was used as the loading control. C-3, C-4: caspase 3 and caspase 4, respectively. M denotes lane with molecular weight marker, and “+” marks lane with a positive control for the respective target antigen.

FIGS. 26A-26C. NEO212 induces protein markers of ER stress and inhibits cell proliferation markers. HUT-78 cells (FIG. 26A), HUT-102 cells (FIG. 26B), and MyLa cells (FIG. 26C) were treated with increasing concentrations of NEO212 or vehicle (vh.). HUT-78 cells (FIG. 26A) and HUT-102 cells (FIG. 26B) were treated for 72 hours, and MyLa cells (FIG. 26C) for 96 hours. In all cases, total cell lysates were prepared and subjected to Western blot analysis with specific antibodies to markers of ER stress (CHOP) and cell proliferation (c-myc and cyclin D). Actin was used as the loading control. M denotes lane with molecular weight marker, and “+” marks lane with a positive control for the respective target antigen.

FIG. 27. NEO212-mediated effects involve reactive oxidants. MyLa cells received 200 and 500 μM AA or 100 and 300 μM b-ME, followed 15 minutes later by the addition of 80 μM NEO212. In parallel, cells were treated with 200 μM H₂O₂. Forty-eight hours later, cells were harvested, and lysates were analyzed by Western blot with specific antibodies. M denotes lane with molecular weight marker and “+” marks lane with a positive control for the respective target antigen. cl. C-3: cleaved (i.e., activated) caspase 3; cl. PARP: cleaved PARP-1.

### DETAILED DESCRIPTION OF THE INVENTION

The present invention provides for a derivative of monoterpene or sesquiterpene, such as a perillyl alcohol derivative. The present invention also provides for a pharmaceutical composition comprising a derivative of monoterpene or sesquiterpene, such as a perillyl alcohol derivative. For example, the perillyl alcohol derivative may be a perillyl alcohol carbamate. The perillyl alcohol derivative may be perillyl alcohol conjugated with a therapeutic agent such as a chemotherapeutic agent. The monoterpene (or sesquiterpene) derivative may be formulated into a pharmaceutical composition, where the monoterpene (or sesquiterpene) derivative is present in amounts ranging from about 0.01% (w/w) to about 100% (w/w), from about 0.1% (w/w) to about 80% (w/w), from about 1% (w/w) to about 70% (w/w), from about 10% (w/w) to about 60% (w/w), or from about 0.1% (w/w) to about 20% (w/w). The present compositions can be administered alone, or may be co-administered together with radiation or another agent (e.g., a chemotherapeutic agent), to treat a disease such as cancer. Treatments may be sequential, with the monoterpene (or sesquiterpene) derivative being administered before or after the administration of other agents. For example, a perillyl alcohol carbamate may be used to sensitize a cancer patient to radiation or chemotherapy. Alternatively, agents may be administered concurrently. The route of administration may vary, and can include, inhalation, intranasal, oral, transdermal, intravenous, subcutaneous or intramuscular injection. The present invention also provides for a method of treating a disease such as cancer, comprising the step of delivering to a patient a therapeutically effective amount of a derivative of monoterpene (or sesquiterpene).

The compositions of the present invention may contain one or more types of derivatives of monoterpene (or sesquiterpene). Monoterpenes include terpenes that consist of two isoprene units. Monoterpenes may be linear (acyclic) or contain rings. Derivatives of monoterpenoids are also encompassed by the present invention. Monoterpenoids may be produced by biochemical modifications such as oxidation or rearrangement of monoterpenes. Examples of monoterpenes and monoterpenoids include, perillyl alcohol (S(−)) and (R(+)), ocimene, myrcene, geraniol, citral, citronellol, citronellal, linalool, pinene, terpineol, terpinen, limonene, terpinenes, phellandrenes, terpinolene, terpinen-4-ol (or tea tree oil), pinene, terpineol, terpinen; the terpenoids such as p-cymene which is derived from monocyclic terpenes such as menthol, thymol and carvacrol; bicyclic monoterpenoids such as camphor, borneol and eucalyptol.

Monoterpenes may be distinguished by the structure of a carbon skeleton and may be grouped into acyclic monoterpenes (e.g., myrcene, (Z)- and (E)-ocimene, linalool, geraniol, nerol, citronellol, myrcenol, geranial, citral a, neral, citral b, citronellal, etc.), monocyclic monoterpenes (e.g., limonene, terpinene, phellandrene, terpinolene, menthol, carveol, etc.), bicyclic monoterpenes (e.g., pinene, myrtenol, myrtenal, verbanol, verbanon, pinocarveol, carene, sabinene, camphene, thujene, etc.) and tricyclic monoterpenes (e.g. tricyclene). See Encyclopedia of Chemical Technology, Fourth Edition, Volume 23, page 834-835.

Sesquiterpenes of the present invention include terpenes that consist of three isoprene units. Sesquiterpenes may be linear (acyclic) or contain rings. Derivatives of sesquiterpenoids are also encompassed by the present invention. Sesquiterpenoids may be produced by biochemical modifications such as oxidation or rearrangement of sesquiterpenes. Examples of sesquiterpenes include farnesol, farnesal, farnesylic acid and nerolidol.

The derivatives of monoterpene (or sesquiterpene) include, but are not limited to, carbamates, esters, ethers, alcohols and aldehydes of the monoterpene (or sesquiterpene). Monoterpene (or sesquiterpene) alcohols may be derivatized to carbamates, esters, ethers, aldehydes or acids.

Carbamate refers to a class of chemical compounds sharing the functional group

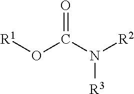

based on a carbonyl group flanked by an oxygen and a nitrogen. R¹, R² and R³ can be a group such as alkyl, aryl, etc., which can be substituted. The R groups on the nitrogen and the oxygen may form a ring. R¹—OH may be a monoterpene, e.g., POH. The R²—N—R³ moiety may be a therapeutic agent.

Carbamates may be synthesized by reacting isocyanate and alcohol, or by reacting chloroformate with amine. Carbamates may be synthesized by reactions making use of phosgene or phosgene equivalents. For example, carbamates may be synthesized by reacting phosgene gas, diphosgene or a solid phosgene precursor such as triphosgene with two amines or an amine and an alcohol. Carbamates (also known as urethanes) can also be made from reaction of a urea intermediate with an alcohol. Dimethyl carbonate and diphenyl carbonate are also used for making carbamates. Alternatively, carbamates may be synthesized through the reaction of alcohol and/or amine precursors with an ester-substituted diaryl carbonate, such as bismethylsalicylcarbonate (BMSC). U.S. Patent Publication No. 20100113819.

Carbamates may be synthesized by the following approach:

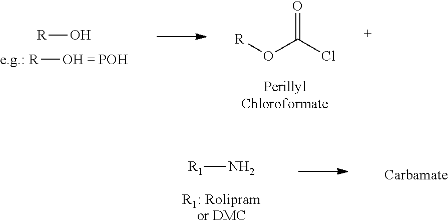

Suitable reaction solvents include, but are not limited to, tetrahydrofuran, dichloromethane, dichloroethane, acetone, and diisopropyl ether. The reaction may be performed at a temperature ranging from about −70° C. to about 80° C., or from about −65° C. to about 50° C. The molar ratio of perillyl chloroformate to the substrate R—NH₂ may range from about 1:1 to about 2:1, from about 1:1 to about 1.5:1, from about 2:1 to about 1:1, or from about 1.05:1 to about 1.1:1. Suitable bases include, but are not limited to, organic bases, such as triethylamine, potassium carbonate, N,N′-diisopropylethylamine, butyl lithium, and potassium-t-butoxide.

Alternatively, carbamates may be synthesized by the following approach:

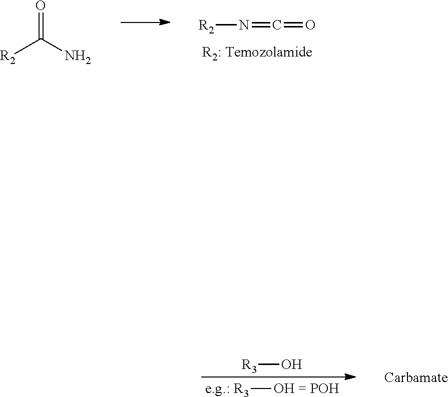

Suitable reaction solvents include, but are not limited to, dichloromethane, dichloroethane, toluene, diisopropyl ether, and tetrahydrofuran. The reaction may be performed at a temperature ranging from about 25° C. to about 110° C., or from about 30° C. to about 80° C., or about 50° C. The molar ratio of perillyl alcohol to the substrate R—N═C═O may range from about 1:1 to about 2:1, from about 1:1 to about 1.5:1, from about 2:1 to about 1:1, or from about 1.05:1 to about 1.1:1.

Esters of the monoterpene (or sesquiterpene) alcohols of the present invention can be derived from an inorganic acid or an organic acid. Inorganic acids include, but are not limited to, phosphoric acid, sulfuric acid, and nitric acid. Organic acids include, but are not limited to, carboxylic acid such as benzoic acid, fatty acid, acetic acid and propionic acid, and any therapeutic agent bearing at least one carboxylic acid functional group Examples of esters of monoterpene (or sesquiterpene) alcohols include, but are not limited to, carboxylic acid esters (such as benzoate esters, fatty acid esters (e.g., palmitate ester, linoleate ester, stearate ester, butyryl ester and oleate ester), acetates, propionates (or propanoates), and formates), phosphates, sulfates, and carbamates (e.g., N,N-dimethylaminocarbonyl). Wikipedia-Ester. Retrieved from URL: http://en.wikipedia.org/wiki/Ester.

A specific example of a monoterpene that may be used in the present invention is perillyl alcohol (commonly abbreviated as POH). The derivatives of perillyl alcohol include, perillyl alcohol carbamates, perillyl alcohol esters, perillic aldehydes, dihydroperillic acid, perillic acid, perillic aldehyde derivatives, dihydroperillic acid esters and perillic acid esters. The derivatives of perillyl alcohol may also include its oxidative and nucleophilic/electrophilic addition derivatives. U.S. Patent Publication No. 20090031455. U.S. Pat. Nos. 6,133,324 and 3,957,856. Many examples of derivatives of perillyl alcohol are reported in the chemistry literature (see Appendix A: CAS Scifinder search output file, retrieved Jan. 25, 2010).

In certain embodiments, a POH carbamate is synthesized by a process comprising the step of reacting a first reactant of perillyl chloroformate with a second reactant such as dimethyl celocoxib (DMC), temozolomide (TMZ) and rolipram. The reaction may be carried out in the presence of tetrahydrofuran and a base such as n-butyl lithium. Perillyl chloroformate may be made by reacting POH with phosgene. For example, POH conjugated with temozolomide through a carbamate bond may be synthesized by reacting temozolomide with oxalyl chloride followed by reaction with perillyl alcohol. The reaction may be carried out in the presence of 1,2-dichloroethane.

POH carbamates encompassed by the present invention include, but not limited to, 4-(bis-N,N′-4-isopropenyl cyclohex-1-enylmethyloxy carbonyl [5-(2,5-dimethyl phenyl)-3-trifluoromethyl pyrazol-1-yl] benzenesulfonamide, 4-(3-cyclopentyloxy-4-methoxy phenyl)-2-oxo-pyrrolidine-1-carboxylic acid 4-isopropenyl cyclohex-1-enylmethyl ester, and (3-methyl 4-oxo-3,4-dihydroimidazo[5,1-d][1,2,3,5]tetrazine-8-carbonyl)carbamic acid-4-isopropenyl cyclohex-1-enylmethyl ester. The details of the chemical reactions generating these compounds are described in the Examples below.

In certain embodiments, perillyl alcohol derivatives may be perillyl alcohol fatty acid esters, such as palmitoyl ester of POH and linoleoyl ester of POH, the chemical structures of which are shown below.

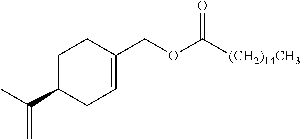

Hexadecanoic acid 4-isopropenyl-cyclohex-1-enylmethyl ester (Palmitoyl ester of POH)

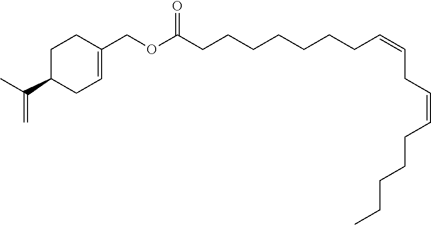 [SMILES: C=C(C)[C@@H]1CC=C(COC(=O)CCCCCCC/C=C\C/C=C\CCCCC)CC1]

Octadeca-9, 12-dienoic acid 4-isopropenyl-cyclohex-1-enylmethyl ester (Linoleoyl ester of POH)

The monoterpene (or sesquiterpene) derivative may be a monoterpene (or sesquiterpene) conjugated with a therapeutic agent. A monoterpene (or sesquiterpene) conjugate encompassed by the present invention is a molecule having a monoterpene (or sesquiterpene) covalently bound via a chemical linking group to a therapeutic agent. The molar ratio of the monoterpene (or sesquiterpene) to the therapeutic agent in the monoterpene (or sesquiterpene) conjugate may be 1:1, 1:2, 1:3, 1:4, 2:1, 3:1, 4:1, or any other suitable molar ratios. The monoterpene (or sesquiterpene) and the therapeutic agent may be covalently linked through carbamate, ester, ether bonds, or any other suitable chemical functional groups. When the monoterpene (or sesquiterpene) and the therapeutic agent are conjugated through a carbamate bond, the therapeutic agent may be any agent bearing at least one carboxylic acid functional group, or any agent bearing at least one amine functional group. In a specific example, a perillyl alcohol conjugate is perillyl alcohol covalently bound via a chemical linking group to a chemotherapeutic agent.

According to the present invention, the therapeutic agents that may be conjugated with monoterpene (or sesquiterpene) include, but are not limited to, chemotherapeutic agents, therapeutic agents for treatment of CNS disorders (including, without limitation, primary degenerative neurological disorders such as Alzheimer's, Parkinson's, multiple sclerosis, Attention-Deficit Hyperactivity Disorder or ADHD, psychological disorders, psychosis and depression), immunotherapeutic agents, angiogenesis inhibitors, and anti-hypertensive agents. Anti-cancer agents that may be conjugated with monoterpene or sesquiterpene can have one or more of the following effects on cancer cells or the subject: cell death; decreased cell proliferation; decreased numbers of cells; inhibition of cell growth; apoptosis; necrosis; mitotic catastrophe; cell cycle arrest; decreased cell size; decreased cell division; decreased cell survival; decreased cell metabolism; markers of cell damage or cytotoxicity; indirect indicators of cell damage or cytotoxicity such as tumor shrinkage; improved survival of a subject; or disappearance of markers associated with undesirable, unwanted, or aberrant cell proliferation. U.S. Patent Publication No. 20080275057.

Also encompassed by the present invention is admixtures and/or coformulations of a monoterpene (or sesquiterpene) and at least one therapeutic agent.

Chemotherapeutic agents include, but are not limited to, DNA alkylating agents, topoisomerase inhibitors, endoplasmic reticulum stress inducing agents, a platinum compound, an antimetabolite, vincalkaloids, taxanes, epothilones, enzyme inhibitors, receptor antagonists, tyrosine kinase inhibitors, boron radiosensitizers (i.e. velcade), and chemotherapeutic combination therapies.

Non-limiting examples of DNA alkylating agents are nitrogen mustards, such as Cyclophosphamide (Ifosfamide, Trofosfamide), Chlorambucil (Melphalan, Prednimustine), Bendamustine, Uramustine and Estramustine; nitrosoureas, such as Carmustine (BCNU), Lomustine (Semustine), Fotemustine, Nimustine, Ranimustine and Streptozocin; alkyl sulfonates, such as Busulfan (Mannosulfan, Treosulfan); Aziridines, such as Carboquone, Triaziquone, Triethylenemelamine; Hydrazines (Procarbazine); Triazenes such as Dacarbazine and Temozolomide (TMZ); Altretamine and Mitobronitol.

Non-limiting examples of Topoisomerase I inhibitors include Campothecin derivatives including SN-38, APC, NPC, campothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as decribed in Pommier Y. (2006) Nat. Rev. Cancer 6(10): 789-802 and U.S. Patent Publication No. 200510250854; Protoberberine alkaloids and derivatives thereof including berberrubine and coralyne as described in Li et al. (2000) Biochemistry 39(24):7107-7116 and Gatto et al. (1996) Cancer Res. 15(12):2795-2800; Phenanthroline derivatives including Benzo[i]phenanthridine, Nitidine, and fagaronine as described in Makhey et al. (2003) Bioorg. Med. Chem. 11 (8): 1809-1820; Terbenzimidazole and derivatives thereof as described in Xu (1998) Biochemistry 37(10):3558-3566; and Anthracycline derivatives including Doxorubicin, Daunorubicin, and Mitoxantrone as described in Foglesong et al. (1992) Cancer Chemother. Pharmacol. 30(2):123-]25, Crow et al. (1994) J. Med. Chem. 37(19):31913194, and Crespi et al. (1986) Biochem. Biophys. Res. Commun. 136(2):521-8. Topoisomerase II inhibitors include, but are not limited to Etoposide and Teniposide. Dual topoisomerase I and II inhibitors include, but are not limited to, Saintopin and other Naphthecenediones, DACA and other Acridine-4-Carboxamindes, Intoplicine and other Benzopyridoindoles, TAS-I03 and other 7H-indeno[2,1-c]Quinoline-7-ones, Pyrazoloacridine, XR 11576 and other Benzophenazines, XR 5944 and other Dimeric compounds, 7-oxo-7H-dibenz[f,ij]Isoquinolines and 7-oxo-7H-benzo[e]pyrimidines, and Anthracenyl-amino Acid Conjugates as described in Denny and Baguley (2003) Curr. Top. Med. Chem. 3(3):339-353. Some agents inhibit Topoisomerase II and have DNA intercalation activity such as, but not limited to, Anthracyclines (Aclarubicin, Daunorubicin, Doxorubicin, Epirubicin, Idarubicin, Amrubicin, Pirarubicin, Valrubicin, Zorubicin) and Antracenediones (Mitoxantrone and Pixantrone).

Examples of endoplasmic reticulum stress inducing agents include, but are not limited to, dimethyl-celecoxib (DMC), nelfinavir, celecoxib, and boron radiosensitizers (i.e. velcade (Bortezomib)).

Platinum based compounds are a subclass of DNA alkylating agents. Non-limiting examples of such agents include Cisplatin, Nedaplatin, Oxaliplatin, Triplatin tetranitrate, Satraplatin, Aroplatin, Lobaplatin, and JM-216. (see McKeage et al. (1997) J. Clin. Oncol. 201:1232-1237 and in general, Chemotherapy for Gynecological Neoplasm, Current Therapy and Novel Approaches, in the Series Basic and Clinical Oncology, Angioli et al. Eds., 2004).

“FOLFOX” is an abbreviation for a type of combination therapy that is used to treat colorectal cancer. It includes 5-FU, oxaliplatin and leucovorin. Information regarding this treatment is available on the National Cancer Institute's web site, cancer.gov.

“FOLFOX/BV” is an abbreviation for a type of combination therapy that is used to treat colorectal cancer. This therapy includes 5-FU, oxaliplatin, leucovorin and Bevacizumab. Furthermore, “XELOX/BV” is another combination therapy used to treat colorectal cancer, which includes the prodrug to 5-FU, known as Capecitabine (Xeloda) in combination with oxaliplatin and bevacizumab. Information regarding these treatments are available on the National Cancer Institute's web site, cancer.gov or from the National Comprehensive Cancer Network's web site, nccn.org.

Non-limiting examples of antimetabolite agents include Folic acid based, i.e., dihydrofolate reductase inhibitors, such as Aminopterin, Methotrexate and Pemetrexed; thymidylate synthase inhibitors, such as Raltitrexed, Pemetrexed; Purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, i.e., cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU). Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S—I (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

Examples of vincalkaloids, include, but are not limited to Vinblastine, Vincristine, Vinflunine, Vindesine and Vinorelbine.

Examples of taxanes include, but are not limited to docetaxel, Larotaxel, Ortataxel, Paclitaxel and Tesetaxel. An example of an epothilone is iabepilone.

Examples of enzyme inhibitors include, but are not limited to farnesyltransferase inhibitors (Tipifarnib); CDK inhibitor (Alvocidib, Seliciclib); proteasome inhibitor (Bortezomib); phosphodiesterase inhibitor (Anagrelide; rolipram); IMP dehydrogenase inhibitor (Tiazofurine); and lipoxygenase inhibitor (Masoprocol). Examples of receptor antagonists include, but are not limited to ERA (Atrasentan); retinoid X receptor (Bexarotene); and a sex steroid (Testolactone).

Examples of tyrosine kinase inhibitors include, but are not limited to inhibitors to ErbB: HER1/EGFR (Erlotinib, Gefitinib, Lapatinib, Vandetanib, Sunitinib, Neratinib); HER2/neu (Lapatinib, Neratinib); RTK class III: C-kit (Axitinib, Sunitinib, Sorafenib), FLT3 (Lestaurtinib), PDGFR (Axitinib, Sunitinib, Sorafenib); and VEGFR (Vandetanib, Semaxanib, Cediranib, Axitinib, Sorafenib); bcr-abl (Imatinib, Nilotinib, Dasatinib); Src (Bosutinib) and Janus kinase 2 (Lestaurtinib).

“Lapatinib” (Tykerb®) is an dual EGFR and erbB-2 inhibitor. Lapatinib has been investigated as an anticancer monotherapy, as well as in combination with trastuzumab, capecitabine, letrozole, paclitaxel and FOLFIRI(irinotecan, 5-fluorouracil and leucovorin), in a number of clinical trials. It is currently in phase III testing for the oral treatment of metastatic breast, head and neck, lung, gastric, renal and bladder cancer.

A chemical equivalent of lapatinib is a small molecule or compound that is a tyrosine kinase inhibitor (TKI) or alternatively a HER-1 inhibitor or a HER-2 inhibitor. Several TKIs have been found to have effective antitumor activity and have been approved or are in clinical trials. Examples of such include, but are not limited to, Zactima (ZD6474), Iressa (gefitinib), imatinib mesylate (STI571; Gleevec), erlotinib (OSI-1774; Tarceva), canertinib (CI 1033), semaxinib (SU5416), vatalanib (PTK787/ZK222584), sorafenib (BAY 43-9006), sutent (SUI 1248) and lefltmomide (SU101).

PTK/ZK is a tyrosine kinase inhibitor with broad specificity that targets all VEGF receptors (VEGFR), the platelet-derived growth factor (PDGF) receptor, c-KIT and c-Fms. Drevs (2003) Idrugs 6(8):787-794. PTK/ZK is a targeted drug that blocks angiogenesis and lymphangiogenesis by inhibiting the activity of all known receptors that bind VEGF including VEGFR-I (Flt-1), VEGFR-2 (KDR/Flk-1) and VEGFR-3 (Flt-4). The chemical names of PTK/ZK are 1-[4-Chloroanilino]-4-[4-pyridylmethyl] phthalazine Succinate or 1-Phthalazinamine, N-(4-chlorophenyl)-4-(4-pyridinylmethyl)-butanedioate (1:1). Synonyms and analogs of PTK/TK are known as Vatalanib, CGP79787D, PTK787/ZK 222584, CGP-79787, DE-00268, PTK-787, PTK787A, VEGFR-TK inhibitor, ZK 222584 and ZK.

Chemotherapeutic agents that can be conjugated with monoterpene or sesquiterpene may also include amsacrine, Trabectedin, retinoids (Alitretinoin, Tretinoin), Arsenic trioxide, asparagine depleter Asparaginase/Pegaspargase), Celecoxib, Demecolcine, Elesclomol, Elsamitrucin, Etoglucid, Lonidamine, Lucanthone, Mitoguazone, Mitotane, Oblimersen, Temsirolimus, and Vorinostat.

The monoterpene or sesquiterpene derivative may be conjugated with angiogenesis inhibitors. Examples of angiogenesis inhibitors include, but are not limited to, angiostatin, angiozyme, antithrombin III, AG3340, VEGF inhibitors, batimastat, bevacizumab (avastin), BMS-275291, CAI, 2C3, HuMV833 Canstatin, Captopril, carboxyamidotriazole, cartilage derived inhibitor (CDI), CC-5013, 6-O-(chloroacetyl-carbonyl)-fumagillol, COL-3, combretastatin, combretastatin A4 Phosphate, Dalteparin, EMD 121974 (Cilengitide), endostatin, erlotinib, gefitinib (Iressa), genistein, halofuginone hydrobromide, Id1, Id3, IM862, imatinib mesylate, IMC-IC11 Inducible protein 10, interferon-alpha, interleukin 12, lavendustin A, LY317615 or AE-941, marimastat, mspin, medroxpregesterone acetate, Meth-1, Meth-2, 2-methoxyestradiol (2-ME), neovastat, oteopontin cleaved product, PEX, pigment epithelium growth factor (PEGF), platelet factor 4, prolactin fragment, proliferin-related protein (PRP), PTK787/ZK 222584, ZD6474, recombinant human platelet factor 4 (rPF4), restin, squalamine, SU5416, SU6668, SU11248 suramin, Taxol, Tecogalan, thalidomide, thrombospondin, TNP-470, troponin-1, vasostatin, VEG1, VEGF-Trap, and ZD6474.

Non-limiting examples of angiogenesis inhibitors also include, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, pentosan polysulfate, angiotensin II antagonists, cyclooxygenase inhibitors (including non-steroidal anti-inflammatory drugs (NSAIDs) such as aspirin and ibuprofen, as well as selective cyclooxygenase-2 inhibitors such as celecoxib and rofecoxib), and steroidal anti-inflammatories (such as corticosteroids, mineralocorticoids, dexamethasone, prednisone, prednisolone, methylpred, betamethasone).

Other therapeutic agents that modulate or inhibit angiogenesis and may also be conjugated with monoterpene or sesquiterpene include agents that modulate or inhibit the coagulation and fibrinolysis systems, including, but not limited to, heparin, low molecular weight heparins and carboxypeptidase U inhibitors (also known as inhibitors of active thrombin activatable fibrinolysis inhibitor [TAFIa]). U.S. Patent Publication No. 20090328239. U.S. Pat. No. 7,638,549.

Non-limiting examples of the anti-hypertensive agents include angiotensin converting enzyme inhibitors (e.g., captopril, enalapril, delapril etc.), angiotensin II antagonists (e.g., candesartan cilexetil, candesartan, losartan (or Cozaar), losartan potassium, eprosartan, valsartan (or Diovan), termisartan, irbesartan, tasosartan, olmesartan, olmesartan medoxomil etc.), calcium antagonists (e.g., manidipine, nifedipine, amlodipine (or Amlodin), efonidipine, nicardipine etc.), diuretics, renin inhibitor (e.g., aliskiren etc.), aldosterone antagonists (e.g., spironolactone, eplerenone etc.), beta-blockers (e.g., metoprolol (or Toporol), atenolol, propranolol, carvedilol, pindolol etc.), vasodilators (e.g., nitrate, soluble guanylate cyclase stimulator or activator, prostacycline etc.), angiotensin vaccine, clonidine and the like. U.S. Patent Publication No. 20100113780.

Other therapeutic agents that may be conjugated with monoterpene (or sesquiterpene) include, but are not limited to, Sertraline (Zoloft), Topiramate (Topamax), Duloxetine (Cymbalta), Sumatriptan (Imitrex), Pregabalin (Lyrica), Lamotrigine (Lamictal), Valaciclovir (Valtrex), Tamsulosin (Flomax), Zidovudine (Combivir), Lamivudine (Combivir), Efavirenz (Sustiva), Abacavir (Epzicom), Lopinavir (Kaletra), Pioglitazone (Actos), Desloratidine (Clarinex), Cetirizine (Zyrtec), Pentoprazole (Protonix), Lansoprazole (Prevacid), Rebeprazole (Aciphex), Moxifloxacin (Avelox), Meloxicam (Mobic), Dorzolamide (Truspot), Diclofenac (Voltaren), Enlapril (Vasotec), Montelukast (Singulair), Sildenafil (Viagra), Carvedilol (Coreg), Ramipril (Delix).

Table 1 lists pharmaceutical agents that can be conjugated with monoterpene (or sesquiterpene), including structure of the pharmaceutical agent and the preferred derivative for conjugation.

TABLE 1

| Brand | Generic | | | Preferred |
|---|---|---|---|---|
| Name | Name | Activity | Structure | Derivative |
| Zoloft | Sertraline | Depression | 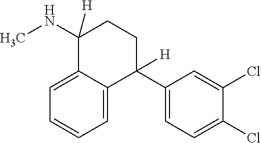 [SMILES: CNC1CCC(c2ccc(Cl)c(Cl)c2)c2ccccc21] | Carbamate |
| Topamax | Topiramate | Seizures | 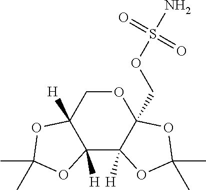 | Carbamate |
| Cymbalta | Duloxetine | Depression | 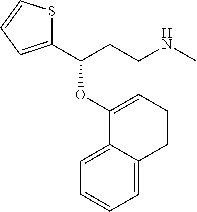 [SMILES: CNCC[C@H](OC1=CCCc2ccccc21)c1cccs1] | Carbamate |
| Imitrex | Sumatriptan | Migraine | 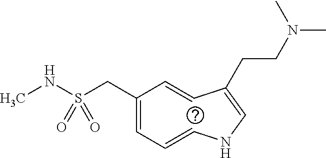 | Carbamate |
| Lyrica | Pregabalin | Neuropathic pain | 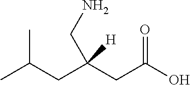 | Carbamate or Ester |
| Lamictal | Lamotrigine | Seizures | 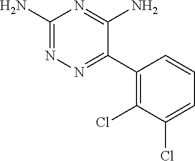 [SMILES: Nc1nnc(-c2cccc(Cl)c2Cl)c(N)n1] | Carbamate |
| Valtrex | Valaciclovir | Herpes | 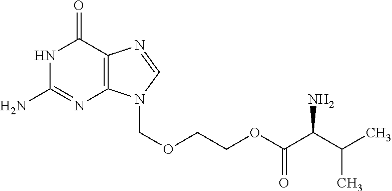 [SMILES: CC(C)[C@H](N)C(=O)OCCOCn1cnc2c(=O)[nH]c(N)nc21] | Carbamate |
| Tarceva | Erlotinib | Non-small cell lung cancer | 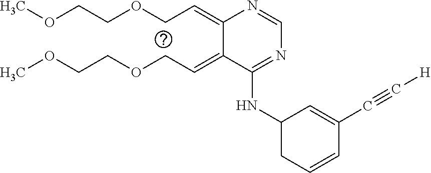 [SMILES: C#CC1=CC(Nc2ncnc(=C/COCCOC)/c2=C\COCCOC)CC=C1] | Carbamate |
| Flomax | Tamsulosin | Benign prostatic Cancer | 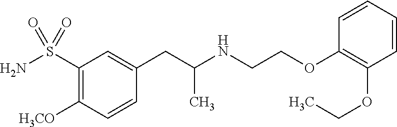 [SMILES: CCOc1ccccc1OCCNC(C)Cc1ccc(OC)c(S(N)(=O)=O)c1] | Carbamate |
| Gleevec | Imatinib | Leukemia | 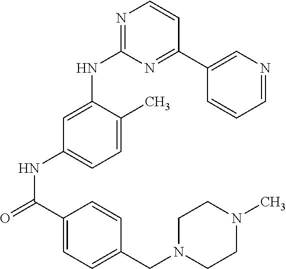 [SMILES: Cc1ccc(NC(=O)c2ccc(CN3CCN(C)CC3)cc2)cc1Nc1nccc(-c2cccnc2)n1] | Carbamate |
| Combivir | Zidovudine | HIV infection | 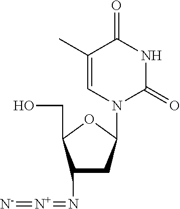 [SMILES: Cc1cn([C@H]2C[C@@H](N=[N+]=[N-])[C@@H](CO)O2)c(=O)[nH]c1=O] | Carbamate |
| Combivir | Lamivudine | HIV infection | 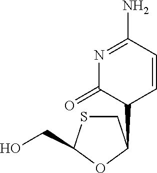 [SMILES: NC1=NC(=O)C([C@@H]2CS[C@H](CO)O2)C=C1] | Carbonate |
| Sustiva | Efavirenz | HIV infection | 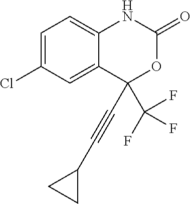 [SMILES: O=C1Nc2ccc(Cl)cc2C(C#CC2CC2)(C(F)(F)F)O1] | Carbamate |
| Epzicom | Abacavir | HIV infection | 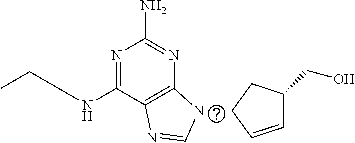 | Carbamate |
| Kaletra | Lopinavir | HIV infection | 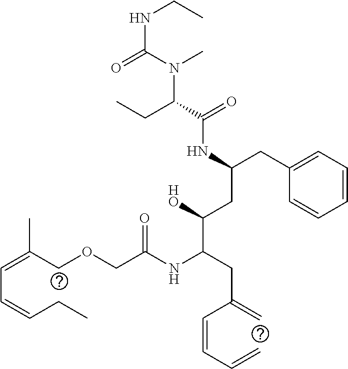 [SMILES: C=C/C=C\C(=C)CC(NC(=O)COC/C(C)=C\C=C/CC)[C@@H](O)C[C@H](Cc1ccccc1)NC(=O)[C@H](CC)N(C)C(=O)NCC] | Carbamate |
| Actos | Pioglitazone | Type-2 diabetes | 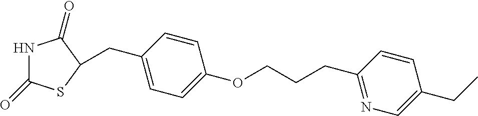 [SMILES: CCc1ccc(CCCOc2ccc(CC3SC(=O)NC3=O)cc2)nc1] | Carbamate |
| Clarinex | Desloratidine | Allergic rhinitis | 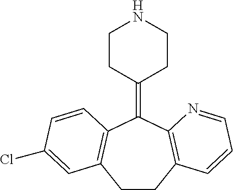 [SMILES: Clc1ccc2c(c1)CCc1cccnc1C2=C1CCNCC1] | Carbamate |
| Zyrtec | Cetirizine | Allergic | 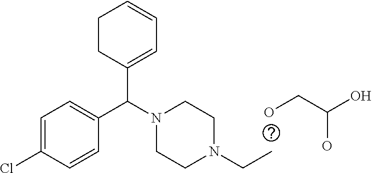 | Ester |
| Protonix | Pentoprazole | Gastrointestinal | 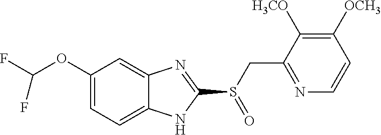 [SMILES: COc1ccnc(C[S@@](=O)c2nc3cc(OC(F)F)ccc3[nH]2)c1OC] | Carbamate |
| Prevacid | Lansoprazole | Gastrointestinal | 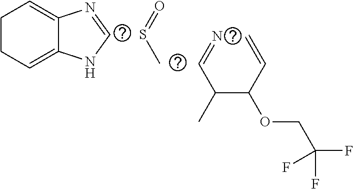 | Carbamate |
| Aciphex | Rebeprazole | Gastrointestinal | 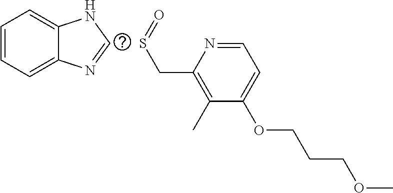 | Carbamate |
| Diovan | Valsartan | Hypertension | 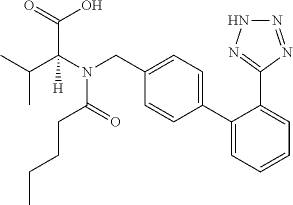 [SMILES: CCCCC(=O)N(Cc1ccc(-c2ccccc2-c2nn[nH]n2)cc1)[C@H](C(=O)O)C(C)C] | Carbamate |
| Cozaar | Losartan | Hypertension | 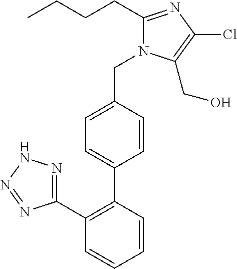 [SMILES: CCCCc1nc(Cl)c(CO)n1Cc1ccc(-c2ccccc2-c2nn[nH]n2)cc1] | Carbamate |
| Avelox | Moxifloxacin | Bacterial infection | 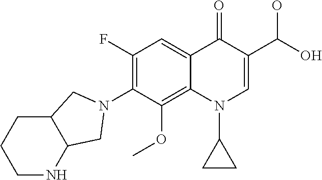 [SMILES: COc1c(N2CC3CCCNC3C2)c(F)cc2c(=O)c(C(O)O)cn(C3CC3)c12] | Carbamate or Ester |
| Mobic | Meloxicam | Osteoarthritis | 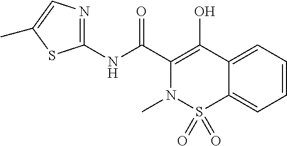 [SMILES: Cc1cnc(NC(=O)C2=C(O)c3ccccc3S(=O)(=O)N2C)s1] | Carbamate |
| Truspot | Dorzolamide | Intraocular pressure | 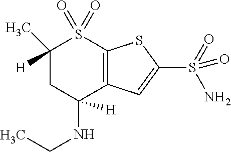 [SMILES: CCN[C@H]1C[C@H](C)S(=O)(=O)c2sc(S(N)(=O)=O)cc21] | Carbamate |
| Voltaren | Diclofenac | Osteoarthritis & rheumatoid arthritis | 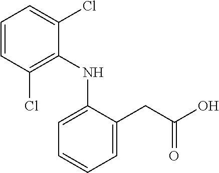 [SMILES: O=C(O)Cc1ccccc1Nc1c(Cl)cccc1Cl] | Carbamate or Ester |
| Vasotec | Enlapril | Hypertension | 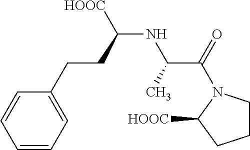 [SMILES: C[C@H](N[C@@H](CCc1ccccc1)C(=O)O)C(=O)N1CCC[C@H]1C(=O)O] | Carbamate or Ester |
| Singulair | Montelukast | Asthma | 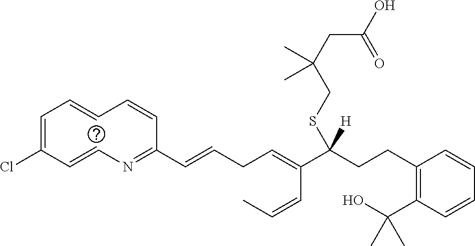 [SMILES: C/C=C\C(=C/C/C=C/c1cccccc(Cl)ccn1)[C@@H](CCc1ccccc1C(C)(C)O)SCC(C)(C)CC(=O)O] | Ester |
| Amlodin | Amlodipine | Hypertension | 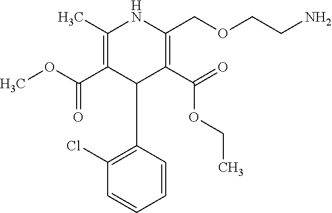 [SMILES: CCOC(=O)C1=C(COCCN)NC(C)=C(C(=O)OC)C1c1ccccc1Cl] | Carbamate |
| Toporol | Metoprolol | Hypertension | 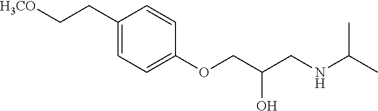 [SMILES: COCCc1ccc(OCC(O)CNC(C)C)cc1] | Carbamate |
| Viagra | Sildenafil | Erectile dysfunction | 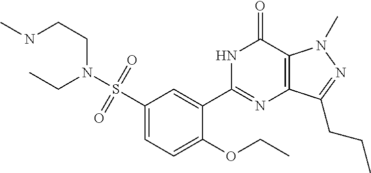 [SMILES: CCCc1nn(C)c2c(=O)[nH]c(-c3cc(S(=O)(=O)N(CC)CCNC)ccc3OCC)nc12] | Carbamate |
| Coreg | Carvedilol | Hypertension | 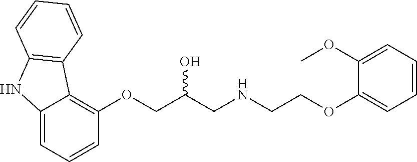 [SMILES: COc1ccccc1OCCNCC(O)COc1cccc2[nH]c3ccccc3c12] | Carbamate |
| Delix | Ramipril | Hypertension | 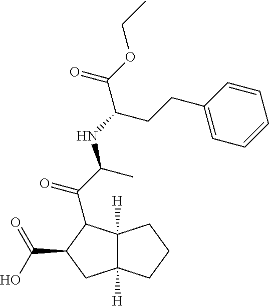 [SMILES: CCOC(=O)[C@H](CCc1ccccc1)N[C@@H](C)C(=O)C1[C@H]2CCC[C@H]2C[C@H]1C(=O)O] | Carbamate or Ester |
| Sinemet (Parcopa, Atamet) | L-DOPA | Neurological disorders | 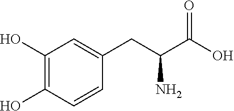 [SMILES: N[C@@H](Cc1ccc(O)c(O)c1)C(=O)O] | |

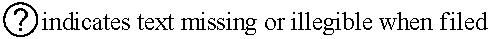

The purity of the monoterpene (or sesquiterpene) derivatives may be assayed by gas chromatography (GC) or high pressure liquid chromatography (HPLC). Other techniques for assaying the purity of monoterpene (or sesquiterpene) derivatives and for determining the presence of impurities include, but are not limited to, nuclear magnetic resonance (NMR) spectroscopy, mass spectrometry (MS), GC-MS, infrared spectroscopy (IR), and thin layer chromatography (TLC). Chiral purity can be assessed by chiral GC or measurement of optical rotation.

The monoterpene (or sesquiterpene) derivatives may be purified by methods such as crystallization, or by separating the monoterpene (or sesquiterpene) derivative from impurities according to the unique physicochemical properties (e.g., solubility or polarity) of the derivative. Accordingly, the monoterpene (or sesquiterpene) derivative can be separated from the monoterpene (or sesquiterpene) by suitable separation techniques known in the art, such as preparative chromatography, (fractional) distillation, or (fractional) crystallization.

The invention also provides for methods of using monoterpenes (or sesquiterpenes) derivatives to treat a disease, such as cancer or other nervous system disorders. A monoterpenes (or sesquiterpenes) derivative may be administered alone, or in combination with radiation, surgery or chemotherapeutic agents. A monoterpene or sesquiterpene derivative may also be co-administered with antiviral agents, anti-inflammatory agents or antibiotics. The agents may be administered concurrently or sequentially. A monoterpenes (or sesquiterpenes) derivative can be administered before, during or after the administration of the other active agent(s).

The monoterpene or sesquiterpene derivative may be used in combination with radiation therapy. In one embodiment, the present invention provides for a method of treating tumor cells, such as malignant glioma cells, with radiation, where the cells are treated with an effective amount of a monoterpene derivative, such as a perillyl alcohol carbamate, and then exposed to radiation. Monoterpene derivative treatment may be before, during and/or after radiation. For example, the monoterpene or sesquiterpene derivative may be administered continuously beginning one week prior to the initiation of radiotherapy and continued for two weeks after the completion of radiotherapy. U.S. Pat. Nos. 5,587,402 and 5,602,184.

Primary cutaneous lymphomas are a heterogenous group of extranodal non-Hodgkin lymphomas. In contrast to nodal non-Hodgkin lymphomas, most of which are B-cell derived, approximately 75% of primary cutaneous lymphomas are T-cell derived.₁ Cutaneous T cell lymphomas (CTCLs) are the most common extranodal non-Hodgkin's T cell lymphomas in adults. Cutaneous T-cell lymphomas (CTCL) are rare and they are characterized by the presence of malignant T-lymphocytes in the skin.^{2,3} They represent 3.9% of all non-Hodgkin lymphomas with an annual incidence of 6.4 to 9.6 cases per million people in the United States.⁴⁻⁶ Most CTCLs fall into three classes: mycosis fungoides, primary cutaneous anaplastic large cell lymphoma (ALCL), and Sezary syndrome.

Mycosis fungoides (MF) and Sézary syndrome (SS) are subtypes of primary cutaneous lymphomas and represent complex diseases regarding their physiopathology and management. Mycosis fungoides (MF) is the most common CTCL, whereas Sézary syndrome (SS) is much rarer. They account for 2-3% of all lymphomas² and comprise approximately 53% of all cutaneous lymphomas.⁴ MF has an annual incidence of 5.6 per million persons³ representing 50% of all CTCL.⁸, whereas SS has an annual incidence of 0.1-0.3 per million persons and represents 2.5% of all CTCL.⁹

Mycosis fungoides (MF), also known as Alibert-Bazin syndrome or granuloma fungoides, is the most common form of cutaneous T-cell lymphoma. Mycosis fungoides is characterized by erythematous patches and plaques (Willemze R. et al. Blood 2005, 105:3768-3785). Symptoms include rash, tumors, skin lesions, and itchy skin. It generally affects the skin, but may progress internally over time. Treatment options include sunlight exposure, ultraviolet light, topical corticosteroids, chemotherapy, and radiotherapy. Depending on the stage of the disease, different treatment regimens are applied. Prognosis for patients with early-stage MF is favorable, but significantly worsens in advanced disease and in SS, where patients frequently relapse and require multiple therapies. Staging is based upon a TNM classification: patients with Stage 1A disease have normal life expectancies, while patients with Stage 1B or greater have a diminished life expectancy (Kim, Y. H. et al. Arch Dermatol 2003, 139:857-866). Patients with Stage II-IV disease have a median survival of less than five years, with large cell transformation often leading to accelerated deterioration (Kim, Y. H. et al. Arch Dermatol 2003, 139:857-866). Sezary syndrome is a leukemic variant of CTCL. Primary cutaneous ALCL has a much less aggressive course, with a five year survival of 95%; however, cutaneous ALCL with concurrent nodal involvement is more aggressive (Willemze R. et al. Blood 2005, 105:3768-3785; Kadin M E, Carpenter C. Semin Hematol 2003, 40:244-256).

The present compounds/compositions and methods may be used to treat, prevent or alleviate a symptom of a primary cutaneous lymphoma. The present compounds/compositions and methods may be used to treat, prevent or alleviate a symptom of an extranodal non-Hodgkin lymphoma or nodal non-Hodgkin lymphoma. The present compounds/compositions and methods may be used to treat, prevent or alleviate a symptom of a hematologic cancer including, but not limited to, cutaneous T-cell lymphoma (CTCL), mycosis fungoides (MF), primary cutaneous anaplastic large cell lymphoma (ALCL), Sezary syndrome, cutaneous B-cell lymphoma, leukemia cutis, or adult T cell leukemia/lymphoma (ATLL).

The present compounds/compositions and methods may be used to treat, prevent or alleviate a symptom of a hematologic cancer including, but not limited to, multiple myeloma, acute lymphocytic leukemia, acute myeloid leukemia, chronic lymphocytic leukemia, small lymphocytic lymphoma, non-Hodgkin's lymphoma, Hodgkin's lymphoma, mantle cell lymphoma, follicular lymphoma, Waldenstrom's macroglobulinemia, B-cell lymphoma and diffuse large B-cell lymphoma, precursor B-lymphoblastic leukemia/lymphoma, B-cell chronic lymphocytic leukemia/small lymphocytic lymphoma, B-cell prolymphocytic leukemia, lymphoplasmacytic lymphoma, splenic marginal zone B-cell lymphoma (with or without villous lymphocytes), hairy cell leukemia, plasma cell myeloma/plasmacytoma, extranodal marginal zone B-cell lymphoma of the MALT type, nodal marginal zone B-cell lymphoma (with or without monocytoid B cells), Burkitt's lymphoma; precursor T-lymphoblastic lymphoma/leukemia, T-cell prolymphocytic leukemia, T-cell granular lymphocytic leukemia, aggressive NK cell leukemia, adult T-cell lymphoma/leukemia (HTLV 1-positive), nasal-type extranodal NK/T-cell lymphoma, enteropathy-type T-cell lymphoma, hepatosplenic gamma-delta T-cell lymphoma, subcutaneous panniculitis-like T-cell lymphoma, anaplastic large cell lymphoma (T/null cell, primary cutaneous type), anaplastic large cell lymphoma (T-/null-cell, primary systemic type), peripheral T-cell lymphoma not otherwise characterized, angioimmunoblastic T-cell lymphoma, polycythemia vera (PV), myelodysplastic syndrome (MDS), indolent Non-Hodgkin's Lymphoma (iNHL) and aggressive Non-Hodgkin's Lymphoma (aNHL).

In one embodiment, the present invention provides for a method of treating tumor cells, such as malignant glioma cells, with chemotherapy, where the cells are treated with an effective amount of a monoterpene derivative, such as a perillyl alcohol carbamate, and then exposed to chemotherapy. Monoterpene derivative treatment may be before, during and/or after chemotherapy.

Monoterpene (or sesquiterpene) derivatives may be used for the treatment of nervous system cancers, such as a malignant glioma (e.g., astrocytoma, anaplastic astrocytoma, glioblastoma multiforme), retinoblastoma, pilocytic astrocytomas (grade I), meningiomas, metastatic brain tumors, neuroblastoma, pituitary adenomas, skull base meningiomas, and skull base cancer. As used herein, the term “nervous system tumors” refers to a condition in which a subject has a malignant proliferation of nervous system cells.

Cancers that can be treated by the present monoterpene (or sesquiterpene) derivatives include, but are not limited to, lung cancer, ear, nose and throat cancer, leukemia, colon cancer, melanoma, pancreatic cancer, mammary cancer, prostate cancer, breast cancer, hematopoietic cancer, ovarian cancer, basal cell carcinoma, biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia including acute myeloid leukemia, acute lymphoid leukemia, chronic myeloid leukemia, chronic lymphoid leukemia; liver cancer; lymphoma including Hodgkin's and Non-Hodgkin's lymphoma; myeloma; fibroma, neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas. U.S. Pat. No. 7,601,355.

The present invention also provides methods of treating CNS disorders, including, without limitation, primary degenerative neurological disorders such as Alzheimer's, Parkinson's, psychological disorders, psychosis and depression. Treatment may consist of the use of a monoterpene or sesquiterpene derivative alone or in combination with current medications used in the treatment of Parkinson's, Alzheimer's, or psychological disorders.

The present invention also provides a method of improving immunomodulatory therapy responses comprising the steps of exposing cells to an effective amount of a monoterpene or sesquiterpene derivative, such as a perillyl alcohol carbamate, before or during immunomodulatory treatment. Preferred immunomodulatory agents are cytokines, such interleukins, lymphokines, monokines, interferons and chemokines.

The present composition may be administered by any method known in the art, including, without limitation, intranasal, oral, transdermal, ocular, intraperitoneal, inhalation, intravenous, ICV, intracisternal injection or infusion, subcutaneous, implant, vaginal, sublingual, urethral (e.g., urethral suppository), subcutaneous, intramuscular, intravenous, rectal, sublingual, mucosal, ophthalmic, spinal, intrathecal, intra-articular, intra-arterial, sub-arachinoid, bronchial and lymphatic administration. Topical formulation may be in the form of gel, ointment, cream, aerosol, etc; intranasal formulation can be delivered as a spray or in a drop; transdermal formulation may be administered via a transdermal patch or iontorphoresis; inhalation formulation can be delivered using a nebulizer or similar device. Compositions can also take the form of tablets, pills, capsules, semisolids, powders, sustained release formulations, solutions, suspensions, elixirs, aerosols, or any other appropriate compositions.

To prepare such pharmaceutical compositions, one or more of monoterpene (or sesquiterpene) derivatives may be mixed with a pharmaceutical acceptable carrier, adjuvant and/or excipient, according to conventional pharmaceutical compounding techniques. Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers and adjuvants, see Remington's Pharmaceutical Sciences, edited by E. W. Martin (Mack Publishing Company, 18th ed., 1990). The compositions also can include stabilizers and preservatives.

As used herein, the term “therapeutically effective amount” is an amount sufficient to treat a specified disorder or disease or alternatively to obtain a pharmacological response treating a disorder or disease. Methods of determining the most effective means and dosage of administration can vary with the composition used for therapy, the purpose of the therapy, the target cell being treated, and the subject being treated. Treatment dosages generally may be titrated to optimize safety and efficacy. Single or multiple administrations can be carried out with the dose level and pattern being selected by the treating physician. Suitable dosage formulations and methods of administering the agents can be readily determined by those of skill in the art. For example, the compositions are administered at about 0.01 mg/kg to about 200 mg/kg, about 0.1 mg/kg to about 100 mg/kg, or about 0.5 mg/kg to about 50 mg/kg. When the compounds described herein are co-administered with another agent or therapy, the effective amount may be less than when the agent is used alone.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer. If the composition is in the form of a gel, the composition may be rubbed onto a membrane of the patient, for example, the skin, preferably intact, clean, and dry skin, of the shoulder or upper arm and or the upper torso, and maintained thereon for a period of time sufficient for delivery of the monoterpene (or sesquiterpene) derivative to the blood serum of the patient. The composition of the present invention in gel form may be contained in a tube, a sachet, or a metered pump. Such a tube or sachet may contain one unit dose, or more than one unit dose, of the composition. A metered pump may be capable of dispensing one metered dose of the composition.

This invention also provides the compositions as described above for intranasal administration. As such, the compositions can further comprise a permeation enhancer. Southall et al. Developments in Nasal Drug Delivery, 2000. The monoterpene (or sesquiterpene) derivative may be administered intranasally in a liquid form such as a solution, an emulsion, a suspension, drops, or in a solid form such as a powder, gel, or ointment. Devices to deliver intranasal medications are well known in the art. Nasal drug delivery can be carried out using devices including, but not limited to, intranasal inhalers, intranasal spray devices, atomizers, nasal spray bottles, unit dose containers, pumps, droppers, squeeze bottles, nebulizers, metered dose inhalers (MDI), pressurized dose inhalers, insufflators, and bi-directional devices. The nasal delivery device can be metered to administer an accurate effective dosage amount to the nasal cavity. The nasal delivery device can be for single unit delivery or multiple unit delivery. In a specific example, the ViaNase Electronic Atomizer from Kurve Technology (Bethell, Wash.) can be used in this invention (http://www.kurvetech.com). The compounds of the present invention may also be delivered through a tube, a catheter, a syringe, a packtail, a pledget, a nasal tampon or by submucosal infusion. U.S. Patent Publication Nos. 20090326275, 20090291894, 20090281522 and 20090317377.

The monoterpene (or sesquiterpene) derivative can be formulated as aerosols using standard procedures. The monoterpene (or sesquiterpene) derivative may be formulated with or without solvents, and formulated with or without carriers. The formulation may be a solution, or may be an aqueous emulsion with one or more surfactants. For example, an aerosol spray may be generated from pressurized container with a suitable propellant such as, dichlorodifluoromethane, trichlorofluoromethane, dichlorotetrafluoroethane, hydrocarbons, compressed air, nitrogen, carbon dioxide, or other suitable gas. The dosage unit can be determined by providing a valve to deliver a metered amount. Pump spray dispensers can dispense a metered dose or a dose having a specific particle or droplet size. As used herein, the term “aerosol” refers to a suspension of fine solid particles or liquid solution droplets in a gas. Specifically, aerosol includes a gas-borne suspension of droplets of a monoterpene (or sesquiterpene), as may be produced in any suitable device, such as an MDI, a nebulizer, or a mist sprayer. Aerosol also includes a dry powder composition of the composition of the instant invention suspended in air or other carrier gas. Gonda (1990) Critical Reviews in Therapeutic Drug Carrier Systems 6:273-313. Raeburn et al., (1992) Pharmacol. Toxicol. Methods 27:143-159.

The monoterpene (or sesquiterpene) derivative may be delivered to the nasal cavity as a powder in a form such as microspheres delivered by a nasal insufflator. The monoterpene (or sesquiterpene) derivative may be absorbed to a solid surface, for example, a carrier. The powder or microspheres may be administered in a dry, air-dispensable form. The powder or microspheres may be stored in a container of the insufflator. Alternatively the powder or microspheres may be filled into a capsule, such as a gelatin capsule, or other single dose unit adapted for nasal administration.

The pharmaceutical composition can be delivered to the nasal cavity by direct placement of the composition in the nasal cavity, for example, in the form of a gel, an ointment, a nasal emulsion, a lotion, a cream, a nasal tampon, a dropper, or a bioadhesive strip. In certain embodiments, it can be desirable to prolong the residence time of the pharmaceutical composition in the nasal cavity, for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl).

The composition containing the purified monoterpene (or sesquiterpene) can be administered by oral inhalation into the respiratory tract, i.e., the lungs.

Typical delivery systems for inhalable agents include nebulizer inhalers, dry powder inhalers (DPI), and metered-dose inhalers (MDI).

Nebulizer devices produce a stream of high velocity air that causes a therapeutic agent in the form of liquid to spray as a mist. The therapeutic agent is formulated in a liquid form such as a solution or a suspension of particles of suitable size. In one embodiment, the particles are micronized. The term “micronized” is defined as having about 90% or more of the particles with a diameter of less than about 10 μm. Suitable nebulizer devices are provided commercially, for example, by PARI GmbH (Starnberg, Germany). Other nebulizer devices include Respimat (Boehringer Ingelheim) and those disclosed in, for example, U.S. Pat. Nos. 7,568,480 and 6,123,068, and WO 97/12687. The monoterpenes (or sesquiterpenes) can be formulated for use in a nebulizer device as an aqueous solution or as a liquid suspension.

DPI devices typically administer a therapeutic agent in the form of a free flowing powder that can be dispersed in a patient's air-stream during inspiration. DPI devices which use an external energy source may also be used in the present invention. In order to achieve a free flowing powder, the therapeutic agent can be formulated with a suitable excipient (e.g., lactose). A dry powder formulation can be made, for example, by combining dry lactose having a particle size between about 1 μm and 100 μm with micronized particles of the monoterpenes (or sesquiterpenes) and dry blending. Alternatively, the monoterpene can be formulated without excipients. The formulation is loaded into a dry powder dispenser, or into inhalation cartridges or capsules for use with a dry powder delivery device. Examples of DPI devices provided commercially include Diskhaler (GlaxoSmithKline, Research Triangle Park, N.C.) (see, e.g., U.S. Pat. No. 5,035,237); Diskus (GlaxoSmithKline) (see, e.g., U.S. Pat. No. 6,378,519; Turbuhaler (AstraZeneca, Wilmington, Del.) (see, e.g., U.S. Pat. No. 4,524,769); and Rotahaler (GlaxoSmithKline) (see, e.g., U.S. Pat. No. 4,353,365). Further examples of suitable DPI devices are described in U.S. Pat. Nos. 5,415,162, 5,239,993, and 5,715,810 and references therein.

MDI devices typically discharge a measured amount of therapeutic agent using compressed propellant gas. Formulations for MDI administration include a solution or suspension of active ingredient in a liquefied propellant. Examples of propellants include hydrofluoroalklanes (HFA), such as 1,1,1,2-tetrafluoroethane (HFA 134a) and 1,1,1,2,3,3,3-heptafluoro-n-propane, (HFA 227), and chlorofluorocarbons, such as CCl₃F. Additional components of HFA formulations for MDI administration include co-solvents, such as ethanol, pentane, water; and surfactants, such as sorbitan trioleate, oleic acid, lecithin, and glycerin. (See, for example, U.S. Pat. No. 5,225,183, EP 0717987, and WO 92/22286). The formulation is loaded into an aerosol canister, which forms a portion of an MDI device. Examples of MDI devices developed specifically for use with HFA propellants are provided in U.S. Pat. Nos. 6,006,745 and 6,143,227. For examples of processes of preparing suitable formulations and devices suitable for inhalation dosing see U.S. Pat. Nos. 6,268,533, 5,983,956, 5,874,063, and 6,221,398, and WO 99/53901, WO 00/61108, WO 99/55319 and WO 00/30614.

The monoterpene (or sesquiterpene) derivative may be encapsulated in liposomes or microcapsules for delivery via inhalation. A liposome is a vesicle composed of a lipid bilayer membrane and an aqueous interior. The lipid membrane may be made of phospholipids, examples of which include phosphatidylcholine such as lecithin and lysolecithin; acidic phospholipids such as phosphatidylserine and phosphatidylglycerol; and sphingophospholipids such as phosphatidylethanolamine and sphingomyelin. Alternatively, cholesterol may be added. A microcapsule is a particle coated with a coating material. For example, the coating material may consist of a mixture of a film-forming polymer, a hydrophobic plasticizer, a surface activating agent or/and a lubricant nitrogen-containing polymer. U.S. Pat. Nos. 6,313,176 and 7,563,768.

The monoterpene (or sesquiterpene) derivative may also be used alone or in combination with other chemotherapeutic agents via topical application for the treatment of localized cancers such as breast cancer or melanomas. The monoterpene (or sesquiterpene) derivative may also be used in combination with narcotics or analgesics for transdermal delivery of pain medication.

This invention also provides the compositions as described above for ocular administration. As such, the compositions can further comprise a permeation enhancer. For ocular administration, the compositions described herein can be formulated as a solution, emulsion, suspension, etc. A variety of vehicles suitable for administering compounds to the eye are known in the art. Specific non-limiting examples are described in U.S. Pat. Nos. 6,261,547; 6, 197,934; 6,056,950; 5,800,807; 5,776,445; 5,698,219; 5,521,222; 5,403,841; 5,077,033; 4,882,150; and 4,738,851.

The monoterpene (or sesquiterpene) derivative can be given alone or in combination with other drugs for the treatment of the above diseases for a short or prolonged period of time. The present compositions can be administered to a mammal, preferably a human. Mammals include, but are not limited to, murines, rats, rabbit, simians, bovines, ovine, porcine, canines, feline, farm animals, sport animals, pets, equine, and primates.

The invention also provides a method for inhibiting the growth of a cell in vitro, ex vivo or in vivo, where a cell, such as a cancer cell, is contacted with an effective amount of the monoterpene (or sesquiterpene) derivative as described herein.

Pathological cells or tissue such as hyperproliferative cells or tissue may be treated by contacting the cells or tissue with an effective amount of a composition of this invention. The cells, such as cancer cells, can be primary cancer cells or can be cultured cells available from tissue banks such as the American Type Culture Collection (ATCC). The pathological cells can be cells of a systemic cancer, gliomas, meningiomas, pituitary adenomas, or a CNS metastasis from a systemic cancer, lung cancer, prostate cancer, breast cancer, hematopoietic cancer or ovarian cancer. The cells can be from a vertebrate, preferably a mammal, more preferably a human. U.S. Patent Publication No. 2004/0087651. Balassiano et al. (2002) Intern. J. Mol. Med. 10:785-788. Thorne, et al. (2004) Neuroscience 127:481-496. Fernandes, et al. (2005) Oncology Reports 13:943-947. Da Fonseca, et al. (2008) Surgical Neurology 70:259267. Da Fonseca, et al. (2008) Arch. Immunol. Ther. Exp. 56:267-276. Hashizume, et al. (2008) Neuroncology 10:112-120.

In vitro efficacy of the present composition can be determined using methods well known in the art. For example, the cytotoxicity of the present monoterpene (or sesquiterpene) and/or the therapeutic agents may be studied by MTT [3-(4,5-dimethylthiazol-2-yl)-2,5-diphenyl tetrazolium bromide] cytotoxicity assay. MTT assay is based on the principle of uptake of MTT, a tetrazolium salt, by metabolically active cells where it is metabolized into a blue colored formazon product, which can be read spectrometrically. J. of Immunological Methods 65: 55 63, 1983. The cytotoxicity of the present monoterpene (or sesquiterpene) derivative and/or the therapeutic agents may be studied by colony formation assay. Functional assays for inhibition of VEGF secretion and IL-8 secretion may be performed via ELISA. Cell cycle block by the present monoterpene (or sesquiterpene) derivative and/or the therapeutic agents may be studied by standard propidium iodide (PI) staining and flow cytometry. Invasion inhibition may be studied by Boyden chambers. In this assay a layer of reconstituted basement membrane, Matrigel, is coated onto chemotaxis filters and acts as a barrier to the migration of cells in the Boyden chambers. Only cells with invasive capacity can cross the Matrigel barrier. Other assays include, but are not limited to cell viability assays, apoptosis assays, and morphological assays.

The following are examples of the present invention and are not to be construed as limiting.

### EXAMPLES

### Example 1: Synthesis of Dimethyl Celecoxib bisPOH Carbamate (4-(bis-N,N′-4-isopropenyl cyclohex-1-enylmethyloxy carbonyl [5-(2,5-dimethyl phenyl)-3-trifluoromethyl pyrazol-1-yl] benzenesulfonamide)

The reaction scheme is the following:

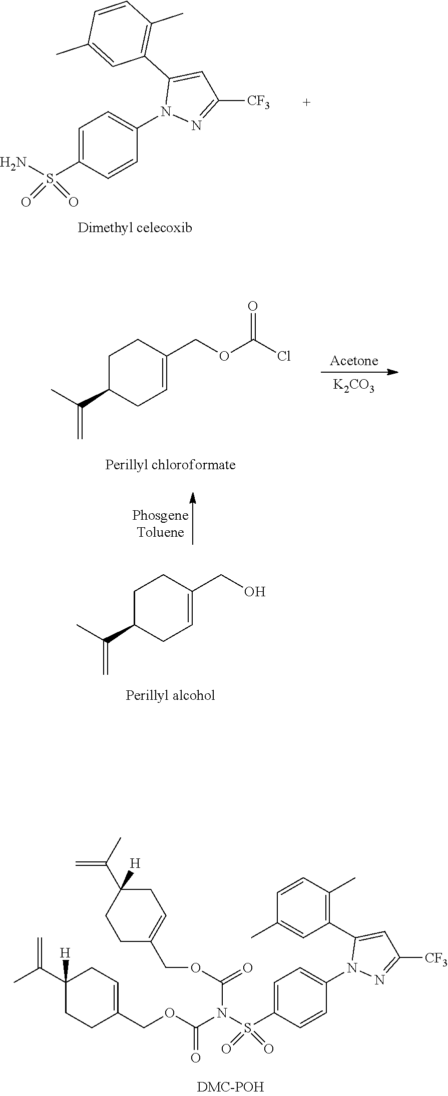

Phosgene (20% in toluene, 13 ml, 26.2 mmol) was added to a mixture of perillyl alcohol (2.0 grams, 13.1 mmol) and potassium carbonate (5.4 grams, 39.1 mmol) in dry toluene (30 mL) over a period of 30 minutes while maintaining the temperature between 10° C. to 15° C. The reaction mixture was allowed to warm to room temperature and stirred for 8.0 hours under N₂. The reaction mixture was quenched with water (30 mL) and the organic layer was separated. The aqueous layer was extracted with toluene (20 mL) and the combined organic layer was washed with water (50 mL×2), brine (15%, 30 mL) and dried over sodium sulfate (20 grams). The filtered organic layer was concentrated under vacuum to give perillyl chloroformate as an oil. Weight: 2.5 grams; Yield: 89%. ¹H-NMR (400 MHz, CDCl₃): δ 1.5 (m, 1H), 1.7 (s, 3H), 1.8 (m, 1H), 2.0 (m, 1H), 2.2 (m, 4H), 4.7 (dd, 4H); 5.87 (m, 1H).

Perillyl chloroformate (0.11 grams, 0.55 mmol) was added slowly to a mixture of dimethyl celecoxib (0.2 grams, 0.50 mmol) and potassium carbonate (0.13 grams, 1.0 mmol) in dry acetone (10 mL) over a period of 5 minutes under N₂. The reaction mixture was heated to reflux and maintained for 3 hours. Since TLC analysis indicated the presence of dimethyl celecoxib (>60%), another 1.0 equivalent of perillyl chloroformate was added and refluxed for an additional 5 hours. The reaction mixture was cooled and acetone was concentrated under vacuum to give a residue.

The resulting residue was suspended in water (15 mL) and extracted with ethyl acetate (3×15 mL). The combined organic layer was washed with water (20 mL) followed by brine (15%, 20 mL) and dried over sodium sulfate. The filtered organic layer was concentrated under vacuum to give a residue which was purified by column chromatography [column dimensions: diameter: 1.5 cm, height: 10 cm, silica: 230-400 mesh] and eluted with hexanes (100 mL) followed by a mixture of hexanes/ethyl acetate (95:5, 100 mL). The hexane/ethyl acetate fractions were combined and concentrated under vacuum to give a gummy mass.

The product POH carbamate exhibited a weight of 120 mg and a yield of 31%. ¹H-NMR (400 MHz, CDCl₃): δ 0.9 (m, 2H), 1.4 (m, 2H), 1.7 (m, 7H*), 1.95 (m, 8H*), 2.1 (m, 4H), 2.3 (s, 3H), 4.4 (d, 2H), 4.7 (dd, 2H), 5.6 (br d, 2H), 6.6 (s, 1H), 7.0 (br s, 1H), 7.12 (d, 1H), 7.19 (d, 1H), 7.4 (d, 2H), 7.85 (d, 2H); MS, m/e: 751.8 (M⁺ 3%), 574.3 (100%), 530.5 (45%), 396 (6%). * N.B. further 2H overlapping from presumed impurity discounted in NMR integration.

### Example 2: In vitro Cytotoxicity Studies of Dimethyl Celecoxib bisPOH Carbamate (POH-DMC)

First cytotoxicity assays were carried out after cells were treated with dimethyl-celecoxib (DMC) alone. FIG. 1 shows the results of the MTT cytotoxicity assays performed on human malignant glioma cells U87, A172 and U251 with DMC alone.

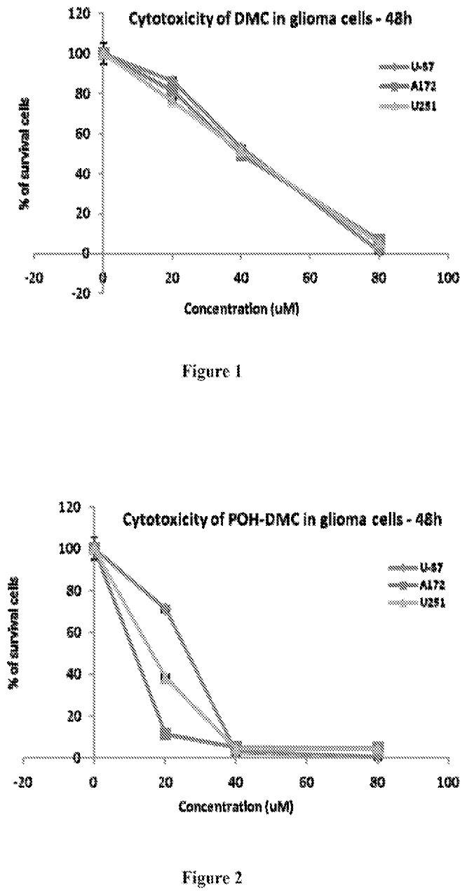

Then U87, A172 and U251 cells were treated with dimethyl celecoxib bisPOH carbamate (POH-DMC) (e.g., synthesized by the method in Example 1), and the MTT cytotoxicity assays performed (FIG. 2). The results suggest that POH carbamate POH-DMC exhibited much better cytotoxicity than DMC alone.

### Example 3: Synthesis of Temozolomide POH Carbamate (3-methyl 4-oxo-3,4-dihydroimidazo[5,1-d][1,2,3,5]tetrazine-8-carbonyl)-carbamic acid-4-isopropenyl cyclohex-1-enylmethyl ester)

The reaction scheme is the following:

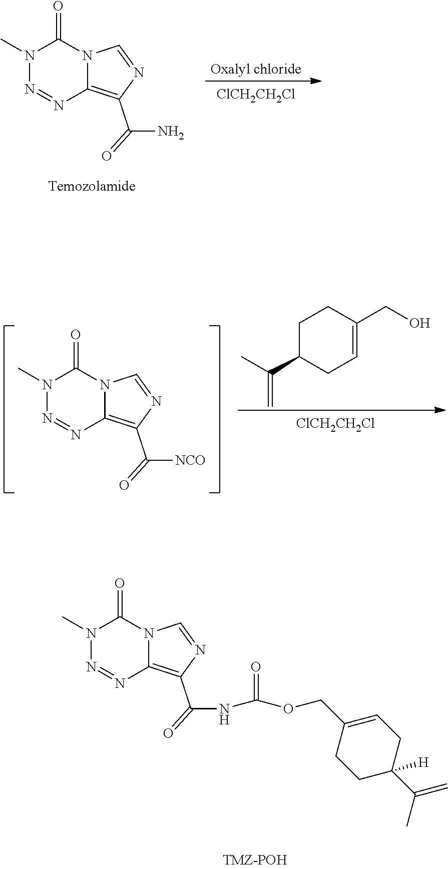

Oxalyl chloride (0.13 grams, 1.0 mmol) was added slowly to a mixture of temozolomide (OChem Incorporation, 0.1 grams, 0.5 mmol) in 1,2-dichloroethane (10 mL) over a period of 2 minutes while maintaining the temperature at 10° C. under N₂. The reaction mixture was allowed to warm to room temperature and then heated to reflux for 3 hours. The excess of oxalyl chloride and 1,2-dichloroethane were removed by concentration under vacuum. The resulting residue was re-dissolved in 1,2-dichlorethane (15 mL) and the reaction mixture was cooled to 10° C. under N₂. A solution of perillyl alcohol (0.086 grams, 0.56 mmol) in 1,2-dichloroethane (3 mL) was added over a period of 5 minutes. The reaction mixture was allowed to warm to room temperature and stirred for 14 hours. 1,2-dichloroethane was concentrated under vacuum to give a residue, which was triturated with hexanes. The resulting yellow solid was filtered and washed with hexanes. Weight: 170 mg; Yield: 89%. ¹H-NMR (400 MHz, CDCl₃): δ 1.4-2.2 (m, 10H), 4.06 (s, 3H), 4.6-4.8 (m, 4H), 5.88 (br s, 1H), 8.42 (s, 1H), 9.31 (br s, 1H); MS, no molecular ion peak was observed. m/e: 314 (100%), 286.5 (17%), 136 (12%).

Alternatively, temozolomide POH carbamate was synthesized according to the following procedure. Oxalyl chloride (0.13 grams, 1.0 mmol) was added slowly to a mixture of temozolomide (OChem Incorporation, 0.1 grams, 0.5 mmol) in 1,2-dichloroethane (10 mL) over a period of 2 minutes while maintaining the temperature at 10° C. under N₂. The reaction mixture was allowed to warm to room temperature and then heated to reflux for 3 hours. The excess of oxalyl chloride and 1,2-dichloroethane were removed by concentration under vacuum. The resulting residue was re-dissolved in 1,2-dichlorethane (15 mL) and the reaction mixture was cooled to 10° C. under N₂. A solution of perillyl alcohol (0.086 grams, 0.56 mmol) in 1,2-dichloroethane (3 mL) was added over a period of 5 minutes. The reaction mixture was allowed to warm to room temperature and stirred for 14 hours. 1,2-Dichloroethane was concentrated under vacuum to give a residue, which was purified by a short silica-plug column (column dimensions: diameter: 2 cm, height: 3 cm, silica: 230-400 mesh) and eluted with a mixture of hexanes/ethyl acetate (1:1, 100 mL). The hexane/ethyl acetate fractions were combined and concentrated under vacuum to give a white solid residue which was triturated with heptanes and filtered to obtain a white solid. Weight: 170 mg; Yield: 89%. ¹H-NMR (400 MHz, CDCl₃): δ 1.4-2.2 (m, 10H), 4.06 (s, 3H), 4.6-4.8 (m, 4H), 5.88 (br s, 1H), 8.42 (s, 1H), 9.31 (br s, 1H); MS, no molecular ion peak was observed, m/e: 314 (100%), 286.5 (17%), 136 (12%).

### Example 4: In Vitro Cytotoxicity Studies of Temozolomide POH Carbamate (POH-TMZ)

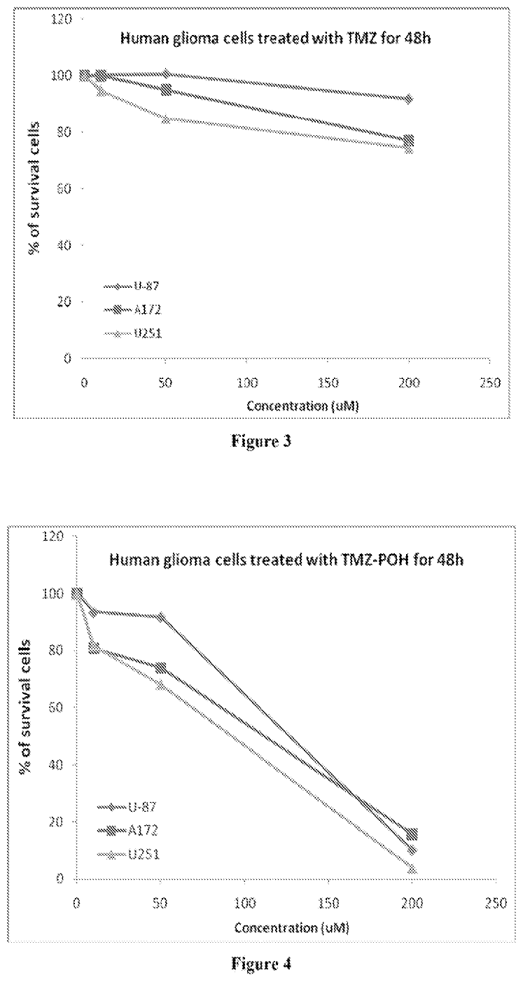

First cytotoxicity assays were carried out after cells were treated with temozolomide (TMZ) alone, the standard alkylating agent used in the treatment of malignant gliomas. FIG. 3 shows the results of the MTT cytotoxicity assays performed on human malignant glioma cells U87, A172 and U251 with TMZ alone. Increasing concentrations of TMZ had minimal cytotoxicity towards the cell lines tested.

Then TMZ-resistant glioma cell lines U87, A172 and U251 cells were treated with temozolomide POH carbamate (POH-TMZ) (e.g., synthesized by the method in Example 3). The MTT assay results (FIG. 4) showed that POH carbamate POH-TMZ exhibited substantially higher kill rates of the various human glioma cells compared to TMZ alone.

### Example 5: Synthesis of Rolipram POH Carbamate (4-(3-cyclopentyloxy-4-methoxy phenyl)-2-oxo-pyrrolidine-1-carboxylic acid 4-isopropenyl cyclohex-1-enylmethyl ester)

The reaction scheme is the following:

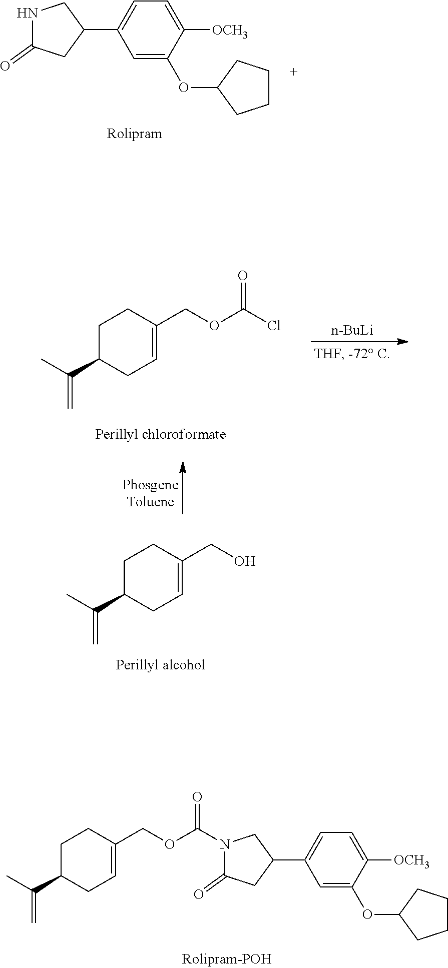

Phosgene (20% in toluene, 13 ml, 26.2 mmol) was added to a mixture of perillyl alcohol (2.0 grams, 13.1 mmol) and potassium carbonate (5.4 grams, 39.1 mmol) in dry toluene (30 mL) over a period of 30 minutes while maintaining the temperature between 10° C. to 15° C. The reaction mixture was allowed to warm to room temperature and stirred for 8.0 hours under N₂. The reaction mixture was quenched with water (30 mL) and the organic layer separated. The aqueous layer was extracted with toluene (20 mL) and the combined organic layer washed with water (50 mL×2), brine (15%, 30 mL) and dried over sodium sulfate (20 grams). The filtered organic layer was concentrated under vacuum to give perillyl chloroformate as an oil. Weight: 2.5 grams; Yield: 89%. ¹H-NMR (400 MHz, CDCl₃): δ 1.5 (m, 1H), 1.7 (s, 3H), 1.8 (m, 1H), 2.0 (m, 1H), 2.2 (m, 4H), 4.7 (dd, 4H); 5.87 (m, 1H).

Butyl lithium (2.5 M, 0.18 mL, 0.45 mmol) was added to a solution of rolipram (GL synthesis, Inc., 0.1 grams, 0.36 mmol) in dry THF at −72° C. over a period of 5 minutes under N₂. After the reaction mixture was stirred for 1.0 hours at −72° C., perillyl chloroformate (dissolved in 4 mL THF) was added over a period of 15 minutes while maintaining the temperature at −72° C. The reaction mixture was stirred for 2.5 hours and quenched with saturated ammonium chloride (5 mL). The reaction mixture was allowed to warm to room temperature and extracted with ethyl acetate (2×15 mL). The combined organic layer was washed with water (15 mL), brine (15%, 15 mL), and then dried over sodium sulfate. The filtered organic layer was concentrated to give an oil which was purified by column chromatography [column dimensions: diameter: 1.5 cm, height: 10 cm, silica: 230-400 mesh] and eluted with a mixture of 8% ethyl acetate/hexanes (100 mL) followed by 12% ethyl acetate/hexanes (100 mL). The 12% ethyl acetate/hexanes fractions were combined and concentrated under vacuum to yield a gummy solid. Weight: 142 mg; Yield: 86%. ¹H-NMR (400 MHz, CDCl₃): δ 1.5 (m, 1H), 1.6 (m, 2H), 1.7 (s, 3H), 1.9 (m, 6H), 2.2 (m, 5H), 2.7 (m, 1H), 2.9 (m, 1H), 3.5 (m, 1H), 3.7 (m, 1H), 3.8 (s, 3H), 4.2 (m, 1H), 4.7 (m, 6H), 5.8 (br s, 1H), 6.8 (m, 3H); MS, m/e: 452.1 (M^{t}′ 53%), 274.1 (100%), 206.0 (55%).

### Example 6: In Vitro Cytotoxicity Studies of Rolipram POH Carbamate (POH-Rolipram)

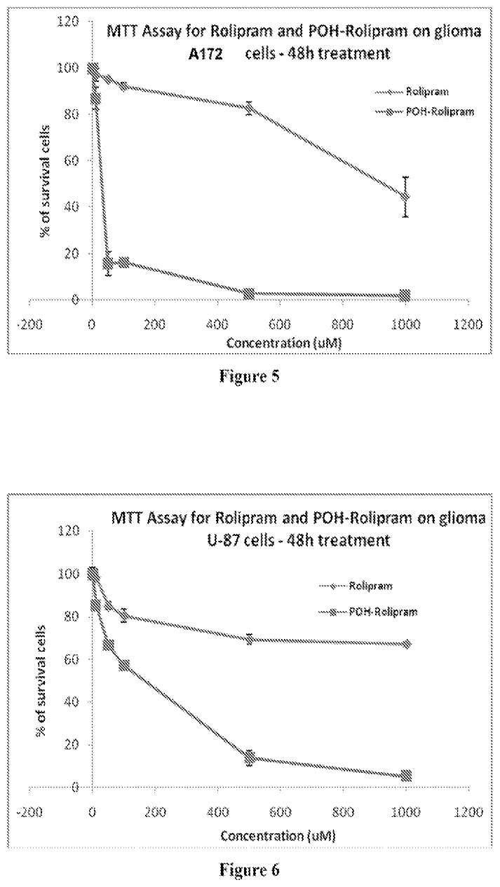

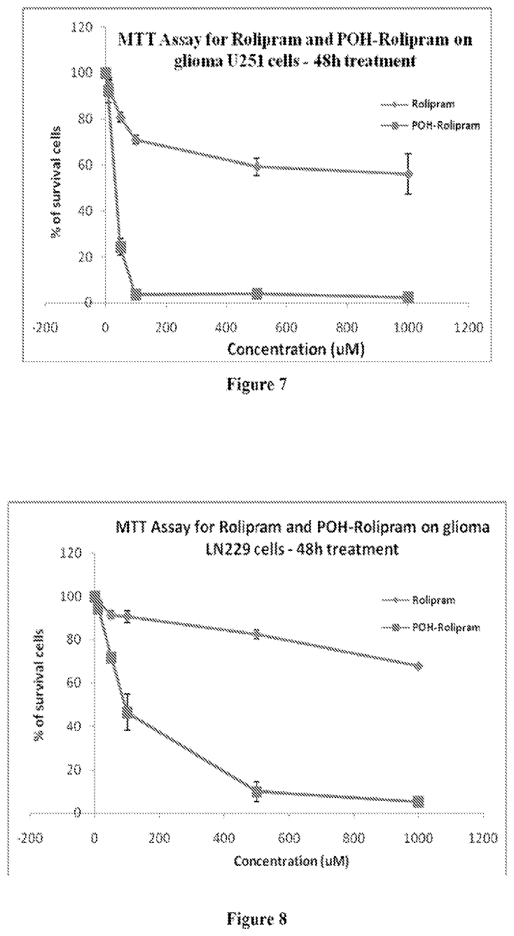

To compare the cytotoxicity of Rolipram POH Carbamate (POH-Rolipram) (e.g., synthesized by the method in Example 5) with rolipram, a type IV phosphodiesterase inducing differentiation and apoptosis in glioma cells, A172, U87, U251 and LN229 human glioma cells were treated with either POH-Rolipram or rolipram for 48 hours. The MTT assay results are shown in FIGS. 5 to 8. POH-Rolipram exhibited substantially higher kill rates compared to rolipram alone for each of the several different human glioma cell types. FIG. 5 shows the MTT assay for increasing concentrations of rolipram and POH-rolipram for A-172 cells. Rolipram alone demonstrates an IC50 of approximately 1000 uM (1 mM). In the presence of POH-rolipram, IC50 is achieved at concentrations as low as 50 uM. FIG. 6 shows the MTT assay for increasing concentrations of rolipram with U-87 cells. IC50 is not met at 1000 uM. On the other hand, IC50 iss achieved at 180 uM with POH-rolipram. FIG. 7 shows that IC50 for rolipram alone for U251 cells is achieved at 170 uM; plateau cytotoxicity is reached at 60%. POH-rolipram achieves IC50 at 50 uM, with almost 100% cytoxicity at 100 uM. FIG. 8 shows that IC50 for rolipram alone for LN229 cells is not achieved even at 100 uM. On the other hand, IC50 for POH-rolipram is achieved at 100 uM, with almost 100% cytotoxicity at 10 uM.

### Example 7: In Vivo Tumor Growth Inhibition by POH Fatty Acid Derivatives

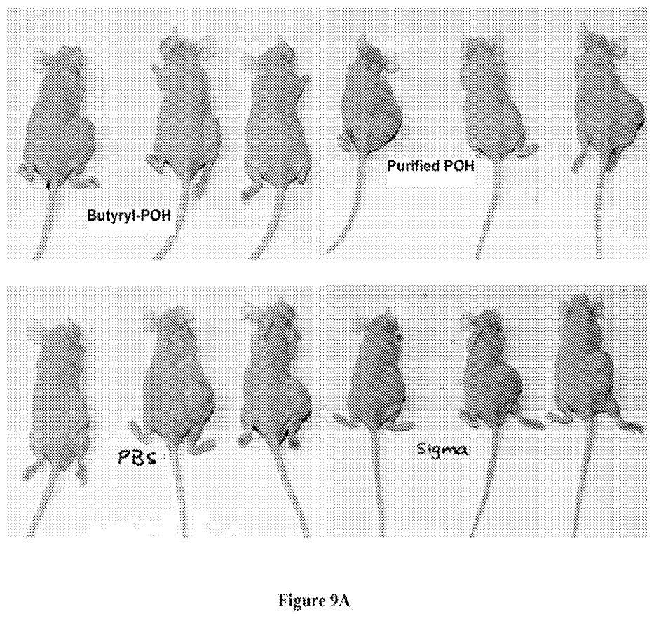

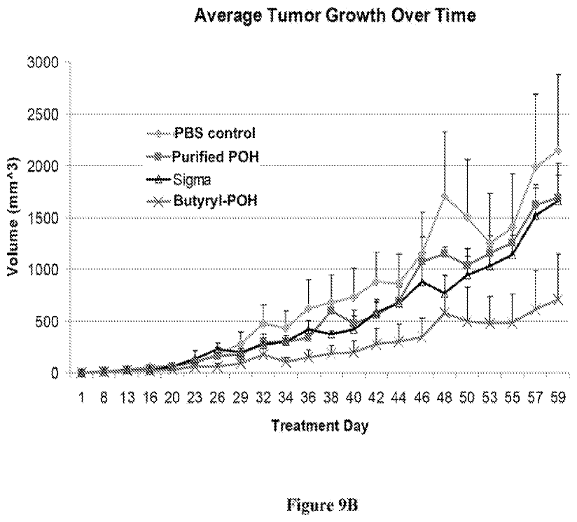

Inhibition of tumor growth by butyryl-POH was studied in a nude mouse subcutaneous glioma model. Mice were injected with U-87 glioma cells (500,000 cells/injection) and allowed to form a palpable nodule over two weeks. Once palpable nodule was formed, the mice were treated with local application of various compounds as indicated in FIGS. 9A and 9B via a Q-tip (1 cc/application/day) over a period of 8 weeks. FIG. 9A shows the images of subcutaneous U-87 gliomas in nude mice treated with butyryl-POH, purified (S)-perillyl alcohol having a purity greater than 98.5% (“purified POH”), POH purchased from Sigma chemicals, or phosphate buffered saline (PBS; negative control). FIG. 9B shows average tumor growth over time (total time period of 60 days). Butyryl-POH demonstrated the greatest inhibition of tumor growth, followed by purified POH and Sigma POH.

### Example 8: In Vitro Cytotoxicity Studies of Temozolomide (TMZ) and Temozolomide POH Carbamate (POH-TMZ) on TMZ Sensitive and Resistant Glioma Cells

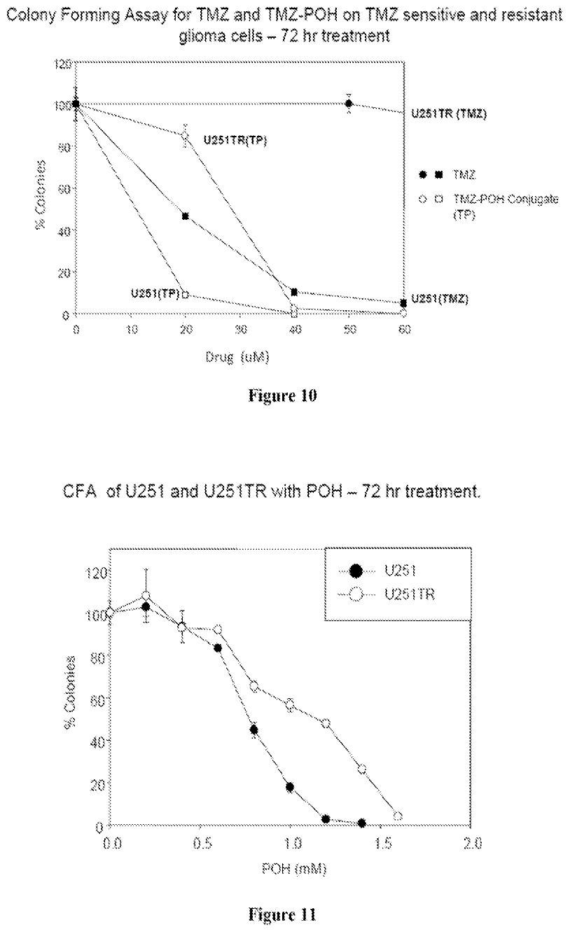

Colony forming assays were carried out after cells were treated with TMZ alone, POH alone, and the TMZ-POH conjugate. The colony forming assays were carried out as described in Chen TC, et al. Green tea epigallocatechin gallate enhances therapeutic efficacy of temozolomide in orthotopic mouse glioblastoma models. Cancer Lett. 2011 Mar. 28; 302(2):100-8. FIG. 10 shows the results of the colony forming assays performed on TMZ sensitive (U251) and TMZ resistant (U251TR) U251 cells with TMZ or TMZ-POH. TMZ demonstrated cytotoxicity towards TMZ sensitive U251 cells, but had minimal cytotoxicity towards TMZ resistant U251 cells. TMZ-POH demonstrated cytotoxicity towards both TMZ sensitive and TMZ resistant U251 cells.

FIG. 11 shows the results of the colony forming assays performed on TMZ sensitive (U251) and TMZ resistant (U251TR) U251 cells with POH. POH demonstrated cytotoxicity towards both TMZ sensitive and TMZ resistant U251 cells. POH-TMZ (FIG. 10) exhibited substantially greater potency compared to POH alone (FIG. 11) in the colony forming assays.

### Example 9: In Vitro Cytotoxicity Studies of Temozolomide POH Carbamate (POH-TMZ) on U251 Cells, U251TR Cells, and Normal Astrocytes

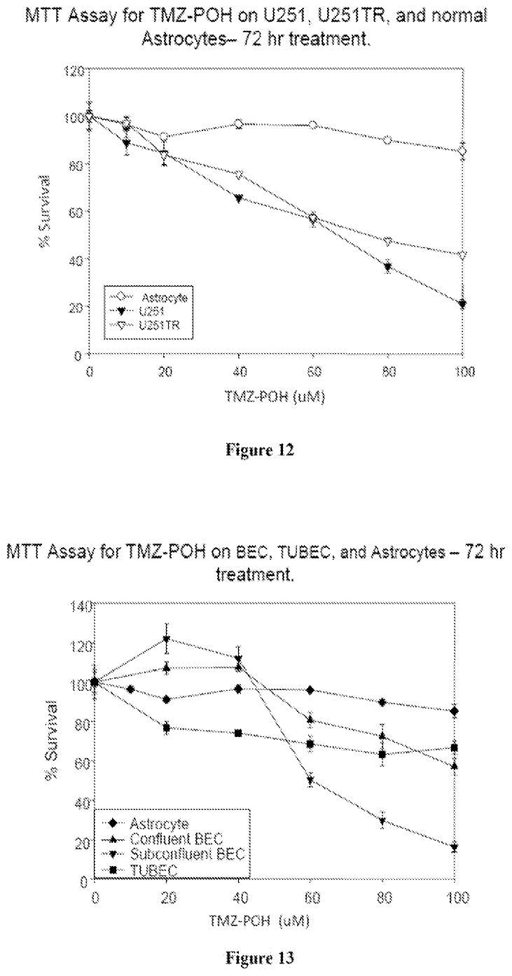

MTT cytotoxicity assays were carried out after cells were treated with the TMZ-POH conjugate. The MTT cytotoxicity assays were carried out as described in Chen TC, et al. Green tea epigallocatechin gallate enhances therapeutic efficacy of temozolomide in orthotopic mouse glioblastoma models. Cancer Lett. 2011 Mar. 28; 302(2):100-8. FIG. 12 shows the results of the MTT cytotoxicity assays performed on TMZ sensitive cells (U251), TMZ resistant cells (U251TR) and normal astrocytes. TMZ-POH demonstrated cytotoxicity towards both TMZ sensitive and TMZ resistant U251 cells, but not towards normal astrocytes.

### Example 10: In Vitro Cytotoxicity Studies of Temozolomide POH Carbamate (POH-TMZ) on BEC, TuBEC, and Normal Astrocytes

MTT cytotoxicity assays were carried out after cells were treated with the TMZ-POH conjugate. The MTT cytotoxicity assays were carried out as described in Chen TC, et al. Green tea epigallocatechin gallate enhances therapeutic efficacy of temozolomide in orthotopic mouse glioblastoma models. Cancer Lett. 2011 Mar. 28; 302(2):100-8. FIG. 13 shows the results of the MTT cytotoxicity assays performed on normal astrocytes, brain endothelial cells (BEC; confluent and subconfluent), and tumor brain endothelial cells (TuBEC). TMZ-POH did not induce significant cytotoxicity on normal astrocytes, confluent BEC, or TuBEC. Mild to moderate cytotoxicity was demonstrated in subconfluent BEC at high concentrations of TMZ-POH.

### Example 11: In Vitro Cytotoxicity Studies of Temozolomide (TMZ) and Temozolomide POH Carbamate (POH-TMZ) on USC-04 Glioma Cancer Stem Cells

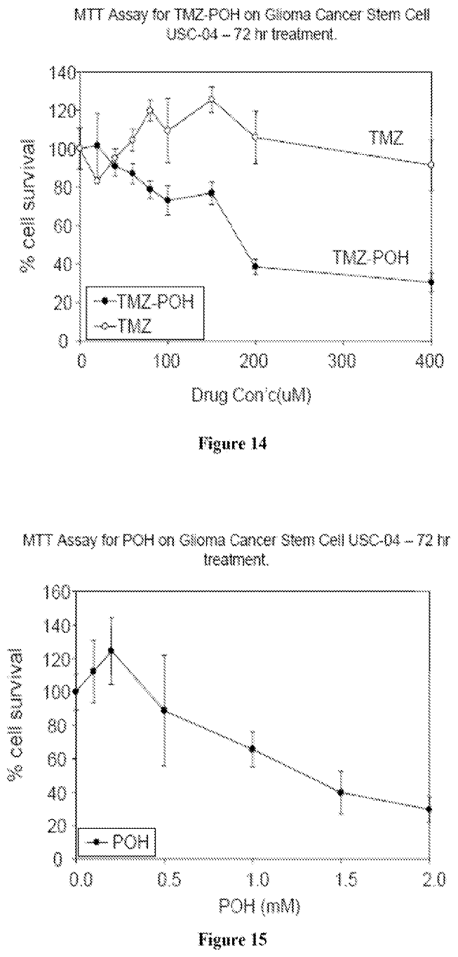

MTT cytotoxicity assays were carried out after cells were treated with the TMZ alone, POH alone, or the TMZ-POH conjugate. The MTT cytotoxicity assays were carried out as described in Chen TC, et al. Green tea epigallocatechin gallate enhances therapeutic efficacy of temozolomide in orthotopic mouse glioblastoma models. Cancer Lett. 2011 Mar. 28; 302(2):100-8. FIG. 14 shows the results of the MTT cytotoxicity assays performed on USC-04 glioma cancer stem cells. TMZ did not induce significant cytotoxicity with increasing concentrations (0-400 uM). TMZ-POH demonstrated evidence of cytotoxicity with IC50 at 150 uM. FIG. 15 shows the results of the MTT cytotoxicity assays performed on USC-04 glioma cancer stem cells treated with POH. POH demonstrated cytotoxicity on USC-04 with increasing concentrations (0-2 mM).

### Example 12: In Vitro Cytotoxicity Studies of Temozolomide (TMZ) and Temozolomide POH Carbamate (POH-TMZ) on USC-02 Glioma Cancer Stem Cells

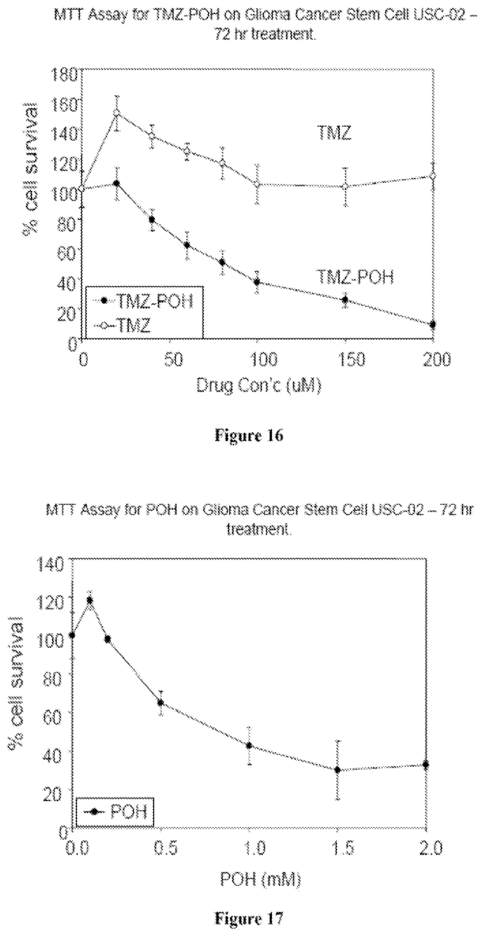

MTT cytotoxicity assays were carried out after cells were treated with the TMZ alone, POH alone, or the TMZ-POH conjugate. The MTT cytotoxicity assays were carried out as described in Chen TC, et al. Green tea epigallocatechin gallate enhances therapeutic efficacy of temozolomide in orthotopic mouse glioblastoma models. Cancer Lett. 2011 Mar. 28; 302(2):100-8. FIG. 16 shows the results of the MTT cytotoxicity assays performed on USC-02 glioma cancer stem cells. TMZ did not induce significant cytotoxicity with increasing concentrations (0-400 uM). TMZ-POH demonstrated evidence of cytotoxicity with IC50 at 60 uM. FIG. 17 shows the results of the MTT cytotoxicity assays performed on USC-02 glioma cancer stem cells treated with POH. POH demonstrated cytotoxicity on USC-02 with increasing concentrations (0-2 mM).

### Example 13: In Vitro Studies of ER Stress by Temozolomide POH Carbamate (POH-TMZ) on

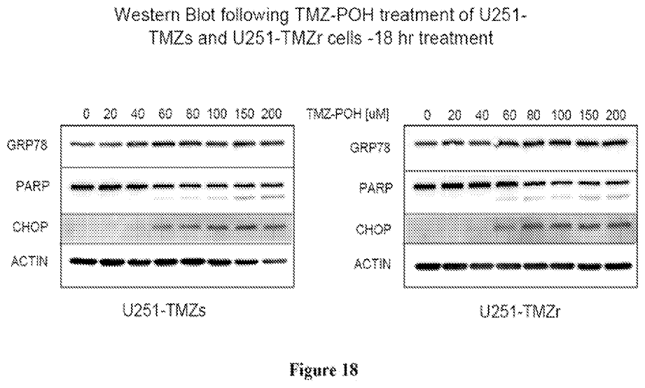

TMZ sensitive and resistant glioma cells Western blots were performed after TMZ sensitive and resistant glioma cells were treated with the TMZ-POH conjugate for 18 hr. FIG. 18 shows a western blot demonstrating that TMZ-POH induces ER stress (ERS) in TMZ sensitive and resistant U251 glioma cells. Activation of the proapoptic protein CHOP was shown at concentrations as low as 60 uM of TMZ-POH.

### Example 14: Use of Temozolomide POH Carbamate (POH-TMZ) to Treat Mycosis Fungoides

HuT 78 cells were purchased from the American Tissue Culture Collection (ATCC TIB-161). HuT 78 cells are cutaneous T lymphocytes derived from a 53-year-old Caucasian patient with Sézary syndrome. The cells were cultured in Iscove's Modified Dulbecco's Medium (IMDM) supplemented with 20% fetal bovine serum.

HuT 78 cells were treated in vitro with (i) temozolomide (50, 100, 250 μM), (ii) POH-TMZ conjugate (“NEO212”, 25, 50, 100 μM), (iii) NEO412 which is the triple conjugate of temozolomide (TMZ), perillyl alcohol (POH), and linoleic acid (25, 50, 100, 250 μM), or (iv) vehicle alone. Seventy-two hours after the addition of drugs or vehicle, cell viability was determined by standard MTT (methylthiazoletetrazolium) assay.

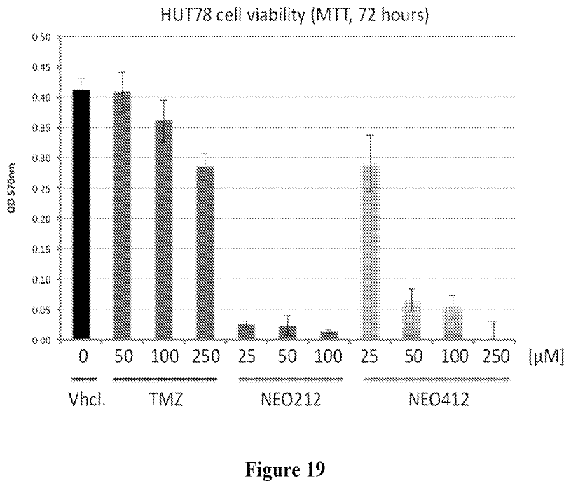

Both the POH-TMZ conjugate (“NEO212”) and the triple conjugate of TMZ, POH and linoleic acid exerted a pronounced cytotoxic effect on the MF cells in vitro. Temozolomide (TMZ) does not show much cytotoxicity on the MF cells (FIG. 19).

Ten million HuT 78 cells were implanted under the skin of an athymic nude mouse. Over the course of the following 10 days, the cells formed a palpable tumor.

Additional mice will be implanted with HuT 78 cells. When palpable tumors have developed, the mice will be treated with systemic NEO212, transdermal NEO412, or vehicle alone as a control.

### Example 15: Cytotoxic Impact of a Perillyl Alcohol-Temozolomide Conjugate on Cutaneous T-Cell Lymphoma In Vitro

Methods: We investigated the potential anticancer effects of NEO212, a compound generated by covalently conjugating perillyl alcohol to temozolomide, on MF and SS cell lines in vitro. HUT-78, HUT-102 and MyLa cells were treated with NEO212 under different conditions, and drug effects on proliferation, viability, and apoptosis were characterized.
Results: NEO212 inhibited proliferation, diminished viability, and stimulated apoptosis in all cell lines, although with varying degrees of potency in the different cell lines. It down-regulated c-myc and cyclin D1 proteins, which are required for cell proliferation, but triggered endoplasmic reticulum stress and activation of caspases. Pre-treatment of cells with anti-oxidants ascorbic acid and beta-mercaptoethanol prevented these NEO212-induced effects.
Conclusions: NEO212 exerted promising anticancer effects on SS and MF cell lines. The generation of reactive oxygen species (ROS) appears to play a key role in the NEO212-induced cell death process, since the blockage of ROS with anti-oxidants prevented caspase activation.
Abbreviations: CTCL: cutaneous T-cell lymphoma; MF: mycosis fungoides; MGMT: 06-methylguanine-DNA methyltransferase; NEO212: perillyl alcohol covalently linked to temozolomide (TMZ-POH); 06-BG: 06-benzylguanine; POH: perillyl alcohol; SS: Sézary syndrome; TMZ: temozolomide.

NEO212 has revealed striking therapeutic activity in a variety of preclinical cancer models, including glioblastoma, melanoma, nasopharyngeal carcinoma, and brain-metastatic breast cancer.¹⁶⁻¹⁹ It is a chimeric molecule that was generated by covalent conjugation of perillyl alcohol (POH) to temozolomide (TMZ). POH, a monoterpene related to limonene, is a natural constituent of caraway, lavender oil, cherries, cranberries, celery seeds, and citrus fruit peel.²⁰ It showed significant anticancer activity in a number of preclinical studies.²¹ Currently ongoing clinical studies with recurrent glioblastoma patients are investigating an intranasal formulation of this compound.

TMZ is an alkylating agent approved for the treatment of newly diagnosed glioblastoma (GBM) and refractory anaplastic astrocytoma.²³ It is also occasionally used for metastatic melanoma and other cancers, but the response rate is low.²⁴ Although TMZ methylates several moieties in different bases of the DNA backbone, it is methylation of the 06-position of guanine (mO6G) that is the decisive toxic lesion that is responsible for triggering subsequent cell death. However, mO6G can be repaired by the DNA repair enzyme 06-methylguanine DNA methyltransferase (MGMT), which removes the methyl group set by TMZ, thereby preventing the cytotoxic sequelae of this lesion. As a result, tumors that express significant levels of MGMT are highly resistant to TMZ therapy.^{25,26}

In our prior work, we studied the anticancer activity of NEO212 in preclinical models and discovered much increased cancer therapeutic potency in vitro and in vivo.¹⁶⁻¹⁹ The promising results obtained so far support the evaluation of its potency and benefit in other difficult-to-treat tumor types. Because of the urgent medical need presented by the lack of effective therapies for CTCL, we performed an in vitro study to investigate the effects of NEO212 in CTCL.

2. Material and methods
2.1. Pharmacological agents—NEO212 was dissolved in DMSO at 100 mM. TMZ was purchased from Sigma Aldrich (St. Louis, Mo.) and dissolved in DMSO (Santa Cruz Biotechnology, Dallas, Tex.) to a concentration of 50 mM. POH was purchased from Sigma-Aldrich and diluted in DMSO to 100 mM. In all cases of cell treatment, the final DMSO concentration in the culture medium never exceeded 1% and was much lower in most cases. Stock solutions of all drugs were stored at −20° C. Staurosporine (STSP) was purchased from Selleck Chemicals (Houston, Tex.), stored at 4° C. protected from light, and dissolved in DMSO before use. Ascorbic acid (AA) and beta-mercaptoethanol (b-ME) (Sigma Aldrich) were prepared fresh before use. Crystalline AA was dissolved in phosphate-buffered saline (PBS) to 25 mM; b-ME was diluted in medium to 25 mM. General 3% household hydrogen peroxide was purchased from CVS Pharmacy and diluted in PBS and medium immediately before its addition to cells.
2.2. Cell lines—Three different human CTLC cell lines were used. HUT78 cells were purchased from the American Tissue Culture Collection (ATCC; Manassas, Va.); this line originated from a patient with Sézary syndrome. HUT-102 also was obtained from the ATCC; this line originated from a patient with mycosis fungoides. MyLa cells originated from a patient with mycosis fungoides. HUT-78 cells were propagated in Iscove's Modified Dulbecco's Medium (IMDM; from VWR, Radnor, Pa., or from ATCC) supplemented with 15% fetal bovine serum (FBS). HUT-102 and MyLa cells were propagated in RPMI medium supplemented with 10% FBS. Both media also contained 100 U/mL penicillin and 0.1 mg/mL streptomycin. Penicillin, streptomycin, and RPMI (prepared with raw materials from Cellgro/MediaTech, Manassas, Va.) were provided by the Cell Culture Core lab of the USC/Norris Comprehensive Cancer Center. HUT-102 cells occasionally received 2 ng/mL interleukin-2 into their medium, although a clear growth benefit did not become apparent. Cells were kept in a humidified incubator at 37° C. and a 5% CO₂ atmosphere. FBS was obtained from Omega Scientific (Tarzana, Calif.) and from X&Y Cell Culture (Kansas City, Mo.). HUT-78 and HUT-102 cells were passaged for less than 6 months after receipt, thus representing authenticated cells.
2.3. MTT assay—Methylthiazoletetrazolium (MTT) assays were performed as follows. Cells were seeded into 96-well plates in a volume of 50 μL per well at 3.0-5.0×10⁵ cells/mL. An additional 50 μL of medium containing various concentrations of drug (or vehicle) was added and the cells were incubated for different lengths of time. This was followed by the addition of 10 μL thiazolyl-blue tetrazolium (i.e., MTT; Sigma Aldrich) from a stock solution of 5 mg/mL in phosphate-buffered saline (PBS). Cells were returned to the incubator for 4 hours. Thereafter, the reaction was stopped and the cells were lysed by the addition of 100 μL solubilization solution (10% sodium dodecyl sulfate in 0.01 M hydrochloric acid). The 96-well plate was left in the cell culture incubator over night for complete solubilization of the MTT crystals, and the optical density (OD) of each well was determined the next day in an ELISA plate reader at 560 nm. The background value (=OD of control wells containing medium without cells+MTT+solubilization solution) was subtracted from all measured values. In individual experiments, each treatment condition was set up in quadruplicate, and each experiment was repeated several times independently.
2.4. Cell proliferation analysis—Cell proliferation was assessed by counting cells over time. Independent cell cultures were exposed to different concentrations of NEO212. At different times, aliquots of cells were removed, mixed with Trypan blue, and counted in a hemocytometer. Blue cells were considered dead, whereas unstained cells were counted as live cells.
2.5. Fluorescence-activated cell sorting (FACS) analysis—Cells were seeded in 6-well tissue culture plates at 2×10⁵ cells/mL, followed by drug treatment. After 72 hours, cells were collected, washed twice with PBS, transferred to a microcentrifuge tube, and suspended in 200 μL of 1× binding buffer solution, which was made fresh from 10x binding buffer (0.2 μm sterile-filtered 0.1 M HEPES, pH 7.4; 1.4 M NaCl; 25 mM CaCl₂)). Then 1 μL of a 1 mg/mL 7-amino-actinomycin D (7-AAD) solution (Thermo Fisher Scientific, Waltham, Mass.) was added. After 20 minutes of incubation on ice, cell fluorescence was analyzed on a FACSAria Flow Cytometer (Becton Dickinson Biosciences Ltd., Franklin Lakes, N.J.).
2.6. Immunoblots—Total cell lysates were prepared by disrupting cells with radio-immunoprecipitation assay (RIPA) buffer²⁸ supplemented with 1 mM PMSF (phenylmethylsulfonyl fluoride, Sigma Aldrich) and Pierce protease inhibitor mini tablets (1 tablet/10 mL; Thermo Fisher Scientific). Protein concentrations were determined using the Pierce BCA protein assay reagent (Thermo Scientific), and 50 μg of total cell lysate from each sample was separated by denaturing polyacrylamide gel electrophoresis (PAGE). Trans-blot (BioRad, Hercules, Calif.) was used for semi-dry transfer to Immobilon-P PVDF membranes (MilliporeSigma, Burlington, Mass.).

We used the following primary antibodies. For the detection of cleaved caspase 3: monoclonal antibody (MAB10753) from MilliporeSigma or monoclonal antibody (SC-271028) from Santa Cruz Biotechnology, Inc. (Dallas, Tex.). For cleaved caspase 4, CHOP, and b-actin: monoclonal antibodies (SC-1229, SC-166682, SC-47778, respectively) from Santa Cruz. For MGMT, c-myc, and cyclin D1: polyclonal antibodies #2739, #13987, and #2922, respectively, from Cell Signaling Technology (Danvers, Mass.). For PARP-1: SC-56196 from Santa Cruz (specific for the cleaved form) and #9542 from Cell Signaling Technology (Danvers, Mass.) (recognizing full-length and cleaved PARP1). Horseradish peroxidase-antibody conjugates (i.e., secondary antibodies) were obtained from Jackson ImmunoResearch Laboratories Inc (West Grove, Pa.). All antibodies were used according to the suppliers' recommendations. For detection, SuperSignal West Pico PLUS Chemiluminescent Substrate was used (Thermo Scientific). Most immunoblots were repeated at least once to confirm the results.

2.7. Statistical analysis—All parametric data were analyzed using Prism software (GraphPad Software, San Diego, Calif.). Student t-tests were applied to calculate the significance values. A probability value (p)<0.05 was considered statistically significant.

### 3. Results

3.1. NEO212 inhibits growth of MF and SS cell lines

NEO212's potential to inhibit the growth of CTCL was investigated in vitro with the use of three established cell lines, HUT78, HUT102, and MyLa. We used two established assays: the standard MTT assay, which primarily indicates cellular metabolic activity and thus viability, and the Trypan blue assay, which directly establishes cell number and thus reveals the proliferative activity of cells.

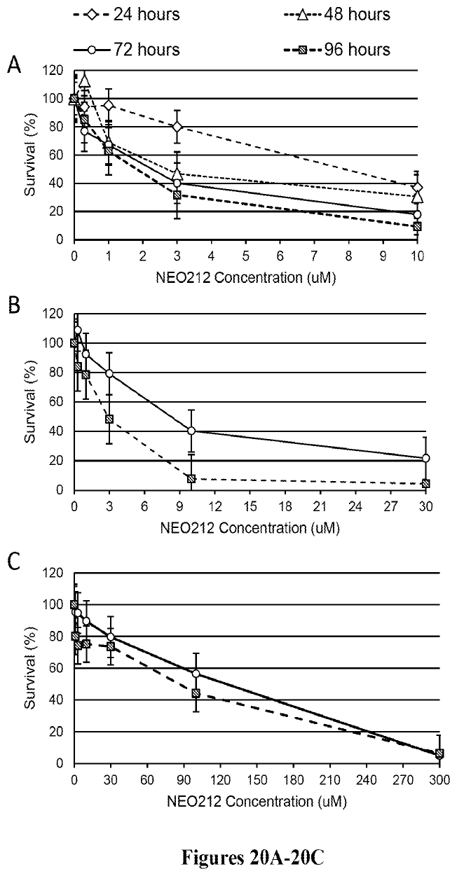

HUT78 cells were exposed to increasing concentrations of NEO212, and MTT assay was performed after 24, 48, 72, and 96 hours. As shown in FIG. 20A, there was a clear time-dependent and concentration-dependent decrease in cellular viability. The earliest effect could be seen at 24 hours with a concentration as low as 3 μM and an IC50 (50% decrease in viability) at 8 μM. At 48 hours, the inhibitory effect of NEO212 became more pronounced, with an IC50 slightly below 3 μM. Longer incubation times, 72 and 96 hours, reduced the IC50 further, although only slightly, as compared to the effects of NEO212 at 48 hours. We therefore chose 72 and 96 hours as the time points for analysis of the other two cell lines. As shown in FIG. 20B, HUT102 cells were somewhat less sensitive to NEO212 as compared to HUT78 cells, with IC50s at 72 and 96 hours of 9 and 3 μM, respectively. In comparison, MyLa cells clearly were the least sensitive cells, with IC50s of about 130 and 85 μM at 72 and 96 hours, respectively (FIG. 20C).

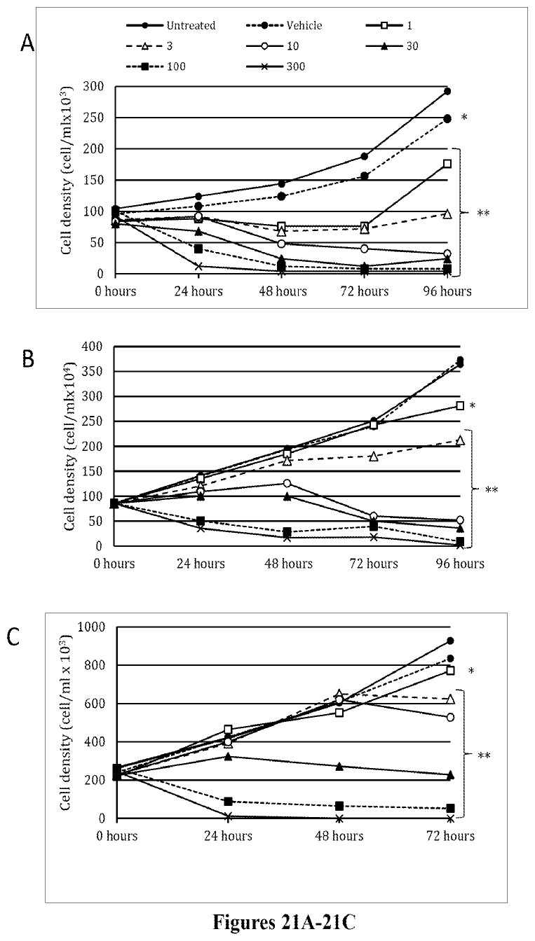

The MTT results were complemented by counting the number of viable cells under different drug concentrations at different time points. Cells were treated with NEO212 at concentrations ranging from 1 to 300 μM, and viable cells (indicated by Trypan blue exclusion) were counted at 24, 48, 72, and 96 hours. Intriguingly, the lowest concentration of NEO212 used, 1 μM, sufficed to exert proliferation-inhibitory effects in all three cell lines, with the strongest effect in HUT78 cells (FIG. 21A), slightly less pronounced activity in HUT102 cells (FIG. 21B), and weaker activity in MyLa cells (FIG. 21C). Higher concentrations of NEO212 exerted correspondingly greater inhibitory activity in all three cell lines, and as before with the MTT assay, HUT78 cells displayed the greatest sensitivity, followed by HUT102 cells. In the case of HUT78 cells, we also noted that vehicle (DMSO) alone exerted minor inhibitory effect. However, this could only be observed at the highest DMSO concentration of 0.3%, which was the one contained in the 300 μM NEO212 dose. Lower concentrations of DMSO did not exert such inhibitory effect, nor was this effect seen in the MTT assays.

Combined, the data from the above MTT and Trypan blue assays show that NEO212 inhibited proliferation and decreased viability in all three CTCL cell lines, although with varying potency. Drug effects on proliferation were generally stronger and highly significant (p<0.01). While MyLa cells appeared more resistant to NEO212 in MTT assays, the Trypan blue exclusion assay revealed a delayed response of these cells to the drug, suggesting that NEO212 might require more time to unfold its inhibitory impact in these cells and trigger their demise.

3.2 NEO212 is More Potent than the Sum of its Parts in HUT78 and Myla Cells

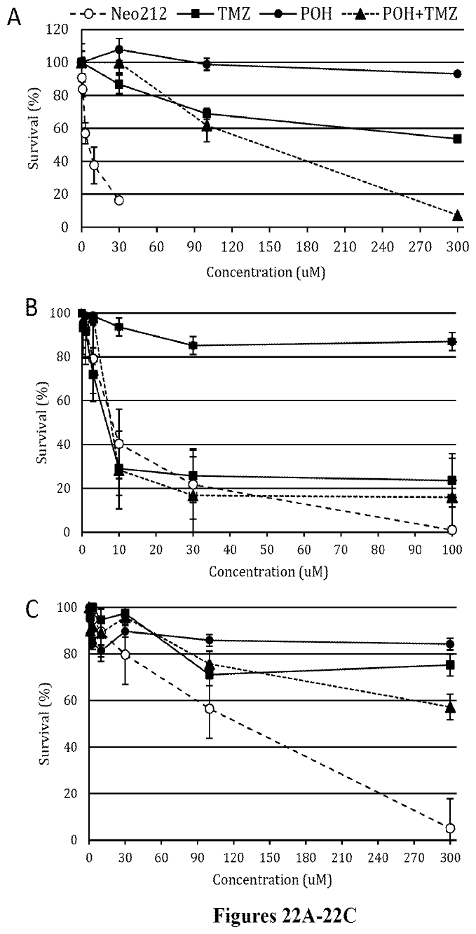

As NEO212 is a chimeric molecule that was generated by covalent conjugation of two anticancer agents, POH and TMZ, we next compared its activity side by side to that of its two constituents, either individually or combined. Cells were treated with increasing concentrations of NEO212, POH alone, TMZ alone, or POH mixed with TMZ, and cell viability was determined by MTT assay after 72 hours. As displayed in FIG. 22A, HUT78 displayed strikingly differential responses to these treatments. As before, NEO212 decreased viability very potently, with an IC50 of about 4 μM. In striking contrast, neither POH nor TMZ reached IC50 at concentrations up to 300 and the combination of POH+TMZ had an IC50 of about 150 μM (i.e., 150 μM POH mixed with 150 μM TMZ).

In HUT102 cells, TMZ alone, as well as the mix of POH+TMZ, yielded similarly potent effects as NEO212 (all in the range of 6-8 μM IC50), whereas POH alone showed very minimal activity (FIG. 22B). In MyLa cells, the IC50 of NEO212 was about 130 whereas none of the other treatments reached IC50 at concentrations up to 300 μM (FIG. 22C). In summary, this analysis revealed that responses in HUT78 and MyLa cells were similar, in that NEO212 was the most potent treatment (although at different IC50 values), whereas all others, including the combination of POH+TMZ, were unable to mimic the potency of NEO212. HUT102 cells however, did not repeat this pattern; rather, these cells displayed similar sensitivity to NEO212, TMZ, and the POH+TMZ combination.

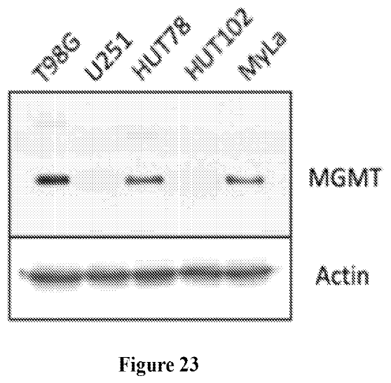

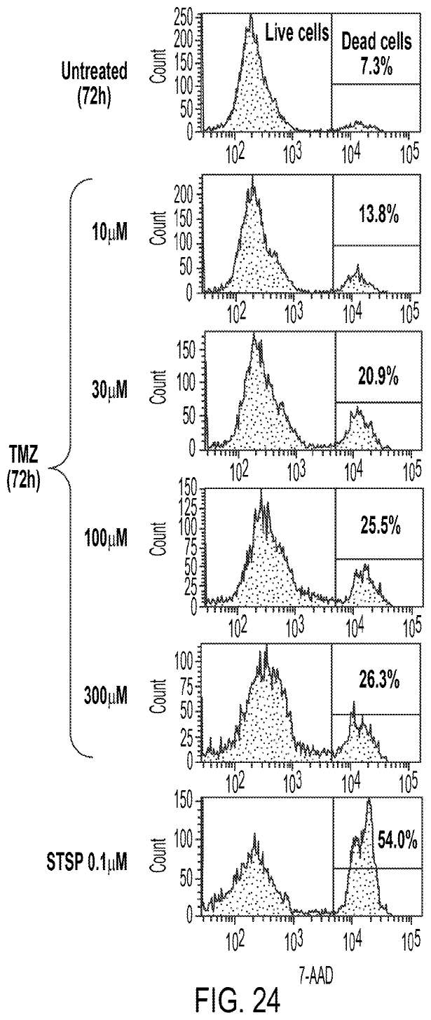

To gain some initial insight as to why HUT102 cells displayed greater sensitivity to TMZ as compared to HUT78 and MyLa cells, we used Western blot analysis to investigate the expression level of MGMT, a DNA repair protein known to confer strong resistance to TMZ.^{25,26} In parallel, we included two established glioblastoma cell lines (TMZ-resistant T98G and TMZ-sensitive U251) as positive and negative controls, respectively. As shown in FIG. 23, HUT78 and MyLa cells presented with prominent MGMT expression similar to T98G cells, whereas HUT102 cells were negative for MGMT, as were U251 cells. Thus, the differential MGMT expression level in the three CTCL cell lines was aligned with their sensitivity to TMZ, but it did not correlate with these cells' sensitivity to NEO212.

### 3.3. NEO212 Causes Apoptotic Cell Death

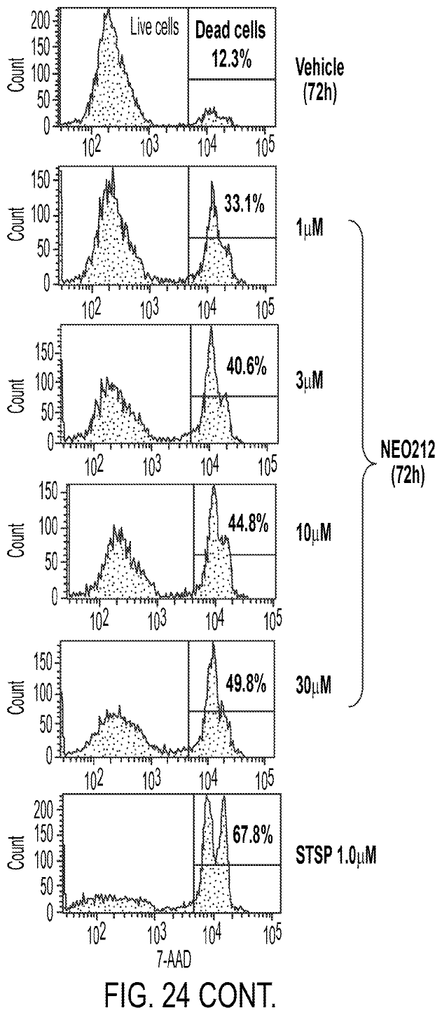

To further characterize the inhibitory effect of NEO212 on CTCL cells, especially in comparison to TMZ, we analyzed drug-induced cell death by FACS analysis with 7-amino-actinomycin D (7-AAD) as a cell death marker. HUT78 cells were treated with 1, 3, 10, and 30 μM NEO212 or with 10, 30, 100, 300 μM TMZ for 72 hours. As a positive control, cells were also treated with staurosporine (STSP), a well-established inducer of apoptotic cell death.²⁹ As displayed in FIG. 24, both NEO212 and TMZ triggered cell death, but NEO212 was substantially more potent. For instance, cell cultures treated with only 1 μM NEO212 showed 33% cell death, whereas the highest concentration of TMZ used, 300 resulted in only 26% cell death. Increasing NEO212 concentrations to 30 μM resulted in 50% cell death, confirming its cell killing potency.

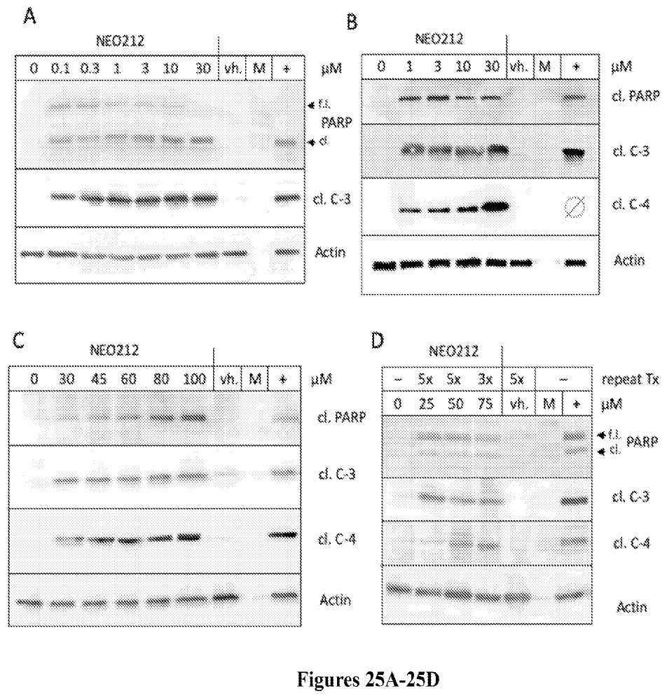

We next investigated established markers of apoptosis, such as cleavage of PARP-1 protein and activation (i.e., cleavage) of caspases. All three CTCL cell lines were treated with increasing concentrations of NEO212, and apoptosis markers were investigated by Western blot analysis. FIGS. 25A-25D shows that treatment with NEO212 resulted in the appearance of cleaved PARP and cleaved (i.e., activated) caspases 3 and 4. The effects were similar in all three CTCL cell lines, except that somewhat higher concentrations of NEO212 were required in MyLa cells to achieve this outcome. Because these latter cells proved to be somewhat less sensitive to NEO212, we further exposed them to repeat daily treatments with NEO212, as would be more relevant for general clinical use in the future. We added 25 or 50 μM NEO212 (or vehicle only) once daily for 5 consecutive days, and cells were harvested 24 hours after the final addition of drug. We also added 75 μM NEO212 on a daily basis, but here we collected cells already after the third treatment, due to obvious, very extensive cell death. As shown in FIG. 25D, repeat treatments also triggered these apoptosis markers, although there was no clear-cut concentration-dependent effect. Because at the time of harvest, all three repeat treatment conditions had caused extensive unhealthy appearances of these cell cultures (as noted by microscopic inspection), we suspect that each condition already resulted in maximal toxic insult. In any case, results shown in FIGS. 25A-25D demonstrate potent induction of apoptotic cell death by NEO212 in all three CTCL cell lines.

### 3.4. NEO212 Induces ER Stress and Cell Cycle Arrest

To gain preliminary insight into mechanisms that might be involved in NEO212-induced apoptosis, we elucidated markers representing three different key processes governing cell fate. The first indicator was CHOP, a central component of the endoplasmic reticulum (ER) stress response that switches the dual mechanism of this response from its pro-survival to its pro-apoptosis mode.³⁰ The second indicator was the protein product of the c-myc proto-oncogene, a mitogenic transcription factor that often is overly active in cancer cells.³¹ The third indicator was cyclin D1, a crucial cell cycle-regulatory component that controls entry into S phase.³²

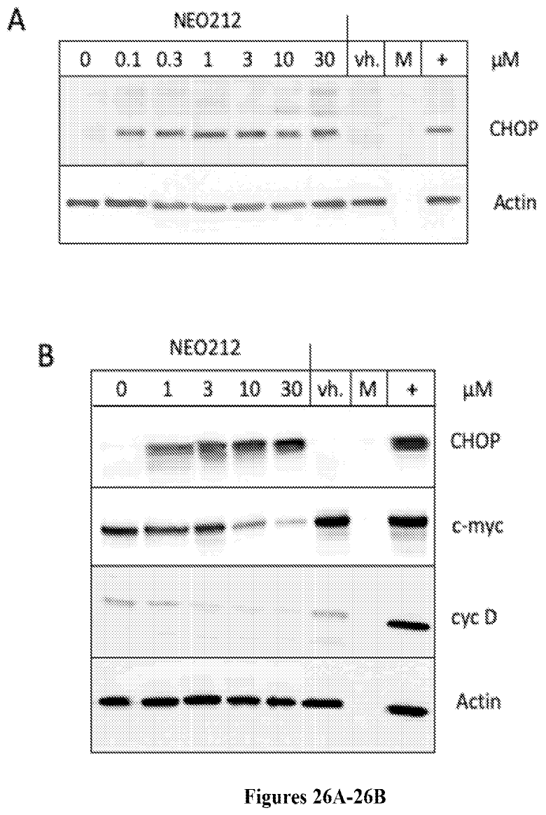

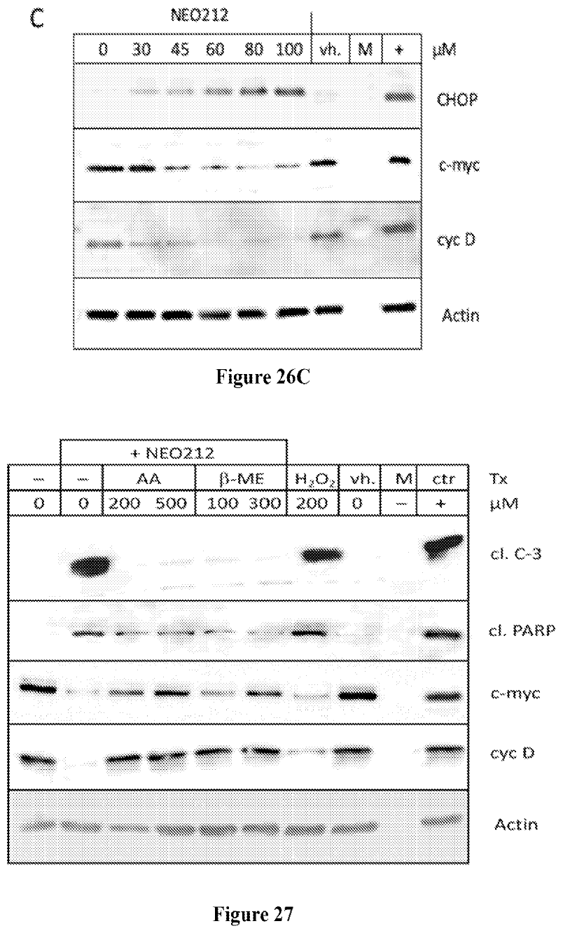

Treatment with NEO212 resulted in prominent induction of CHOP protein in all three CTCL cell lines, indicating the presence of ER stress. Intriguingly, the lowest NEO212 concentrations applied to HUT78 and HUT102 cells, 0.1 μM and 1.0 μM, respectively, sufficed to trigger near-maximal induction of this ER stress indicator (FIGS. 26A-26B), whereas in MyLa cells CHOP induction was substantially more concentration dependent, with a gradual increase all the way up to 100 μM NEO212 (FIG. 26C). Conversely, expression levels of c-myc and cyclin D proteins declined in response to NEO212 treatment of HUT102 and MyLa cells (HUT78 cells were not tested). Together, these results reveal the emergence of pro-apoptotic ER stress in NEO212-treated cells, along with downregulation of a key mitogenic transcriptional stimulator, and inhibition of a component that is required for cell cycle progression. In concert, these events may provide a basis for the observed growth inhibition and apoptosis of NEO212-treated cells.

### 3.5. NEO212 Effects are Dependent on ROS Production

The generation of reactive oxygen species (ROS) plays a role in chemotherapy of several anticancer drugs.³³ We therefore investigated whether ROS might be involved in the above-described effects of NEO212, by including two commonly used anti-oxidants, ascorbic acid (AA) and beta-mercaptoethanol (b-ME).^{34,34} MyLa cells were treated with NEO212 in the presence or absence of AA or b-ME, followed by analysis of the expression levels of c-myc and cyclin D1 (proliferation markers), and activated caspase-3 and cleaved PARP-1 (apoptosis markers). FIG. 27 shows that AA and b-ME exerted striking effects, in that both agents prevented the anticancer impact of NEO212 on the selected marker proteins. In the presence of anti-oxidants, NEO212's prominent activation of caspase-3 was effectively blocked, and cleavage of PARP-1 was significantly diminished. Conversely, down-regulation of c-myc and cyclin D1 by NEO212 was prevented.

These results indicated that the growth-inhibitory and pro-apoptotic effects of NEO212 in these cells were largely mediated by drug-induced generation of ROS. To lend further support to this notion, we also treated cells with H₂O₂, to determine whether NEO212 effects on these same markers as above could be mimicked by directly supplying cells with ROS. As shown in FIG. 27, this was indeed the case, as treatment of cells with H₂O₂ resulted in clear down-regulation of c-myc and cyclin D1 proteins, along with strong activation of caspase-3 and cleavage of PARP-1.

### 4. Discussion

Mycosis fungoides and Sézary syndrome are complex diseases and difficult to manage. Physicians usually have to resort to the use of multiple therapies, and the situation becomes even more challenging in patients with advanced disease.^{1,3} During early stages, skin-directed therapies, such as high-potency topical steroids, topical retinoids and rexinoids, topical nitrogen mustard, and phototherapy, represent first-line regimens with complete response rates ranging from 60 and 100%.^{9,14,15} For patients at early stages who failed topical therapies, physicians can start using combinations with biologic agents, such as interferon alfa, retinoids (all-trans retinoic acid, isotretinoin), rexinoids (bexarotene) and methotrexate. Local radiation therapy is considered in patients with unifocal transformation, isolated/localized cutaneous tumors, or chronic and/or painful and/or ulcerated lesions. Extensive radiation therapies, such as total skin electron beam therapy (TSEBT), is generally reserved for elderly patients or patients with rapidly progressing or refractory widespread plaques and tumors.⁴ At advanced stages of the disease, systemic therapy becomes necessary, but there is no standard regimen for these patients. A variety of approved and unapproved agents are used in these cases, including immune modulators and antibodies as single agents or as combination chemotherapy, or other investigational agents. The current FDA-approved agents for the treatment of CTCL are bexarotene, vorinostat, denileukin diftitox (discontinued in the United States), romidepsin, brentuximab vedotin and mogamulizumab.³⁶ Despite these options, the need for additional and more effective therapeutic agents remains.

A few prior reports provided evidence that alkylating agents provided some benefit for patients with MF or SS. For example, topical carmustine (bis-chloroethylnitrosourea, BCNU) has been used for patch- and plaque-stage MF.³⁷ Oral TMZ has been investigated in two Phase II clinical trials with heavily pre-treated, advanced-stage CTCL patients. The response rate was 33%³⁸ and 27%,³⁹ revealing moderate activity that compared favorably with other treatments. TMZ was also tested in four MF patients with CNS involvement, where it showed moderate activity as well⁴⁰. These results inspired us to investigate NEO212 in MF/SS. Our prior studies with NEO212 established its potent anticancer activity, along with low toxicity, in a variety of preclinical tumor models.¹⁶⁻¹⁹ Although NEO212's therapeutic activity is at least in part based on DNA alkylation (derived from its TMZ component), the covalent conjugation to POH appears provide additional benefits, altogether resulting in significantly greater activity as compared to TMZ.¹⁸ We therefore hypothesized that the promising, but moderate, activity of TMZ in MF/SS, as documented in three clinical studies, can be significantly improved with the use of NEO212.

When compared side-by-side in vitro, NEO212 exerted greater cytotoxic potency than TMZ in all three CTCL cell lines tested (FIGS. 22A-22C). Two of these cell lines (HUT78 and MyLa) essentially were unresponsive to TMZ, as IC50 was not reached at concentrations up to 300 μM. To put these numbers into a physiological context: peak plasma levels of TMZ in cancer patients have been measured in the range of 50-70 μM.^{41,42} In comparison, HUT78 cells turned out to be exquisitely sensitive to NEO212, with concentrations as low as 1 μM NEO212 exerting significant (p<0.01) growth-inhibitory effects (FIGS. 21A-21C), thus revealing a potency that was over 100-fold greater than that of TMZ (FIGS. 22A-22C and 24). The particularly strong effects of NEO212 in HUT78 cells were the more impressive because these cells showed prominent expression of MGMT (FIG. 23). MGMT is well known to confer powerful resistance to TMZ,^{25,26} and unsurprisingly the two MGMT-positive cell lines, HUT78 and MyLa, were unresponsive to TMZ. Yet NEO212 did not follow this pattern: while effective in all three cell lines, its greatest potency was exerted in an MGMT-positive cell line. Altogether, these in vitro results indicate that NEO212 might overcome some of the therapeutic limitations of TMZ, in particular TMZ's ineffectiveness in patients with MGMT-positive tumors, which is well established in the case of malignant glioma.⁴³⁻⁴⁵

It is noteworthy that a mere mix of TMZ and POH at equimolar concentrations was unable to mimic the high potency of NEO212 in HUT78 or MyLa cells (FIGS. 22A-22C). There are a number of studies that have established the anticancer potential of POH in a variety of preclinical studies (see detailed references in ref.²¹). In all cases, fairly high concentrations of this natural monoterpene, usually in the high micromolar to low millimolar range, were required to exert growth-inhibitory or apoptosis-inducing effects in cell culture. For example, several studies with glioblastoma, breast cancer, or melanoma cell lines reported IC50s of 700-1,800 μM of POH in various in vitro cytotoxicity assays.^{16,17,46} Consistent with these earlier reports, our treatments with up to 300 μM POH show only negligible measurable impact on the three CTCL cell lines used (FIGS. 22A-22C). Accordingly, at concentrations up to 100 μM, the addition of POH to TMZ was unable to further enhance the cytotoxic impact over that of TMZ alone. For example, in HUT78 cells 100 μM TMZ alone reduced viability by about 30%, and treatment of cells with 100 μM TMZ in combination with 100 μM POH did not significantly further enhance this inhibitory effect. In comparison, 10 μM NEO212 reduced viability by over 50% (FIG. 3A). This example illustrates that NEO212's potency is greater than the sum of its parts.

It is not entirely clear why covalent conjugation of TMZ to POH, as in NEO212, yields anticancer outcomes that are significantly greater than a mere mix of these two components. It is noteworthy that the cytotoxic impact of NEO212 treatment can be detected earlier than that of TMZ. The alkylating function of TMZ, in particular its methylation of the 06-position of guanine, generally requires two rounds of cell cycle progression in order to generate double-strand DNA breaks and subsequent cytotoxicity.^{26,47} In contrast, cell growth-inhibitory effects of NEO212 can be detected within the first 24 hours (FIGS. 21A-21C), which is more similar to the rapid cytotoxic impact of POH (although substantially higher concentrations of POH are required, as discussed above). These observations suggest that the inherent anticancer activity of NEO212 appears to involve more than DNA alkylation and that possibly POH-based activities are enhanced within its context of the NEO212 molecule.

Among the antitumor functions of POH is its ability to trigger cytotoxic ER stress, which has been demonstrated in glioblastoma cells in vitro.⁴⁶ In general, the cellular ER stress response represents an adaptive mechanism by which the cell attempts to adjust to arising detrimental conditions, such as hypoxia, low nutrient levels, or certain pharmacological agents.⁴⁸ However, if homeostasis cannot be re-established, the pro-apoptotic module of this mechanism, in particular its key executor protein CHOP, gains dominance and tips the balance toward cell death.³⁰ Our finding that NEO212 treatment of CTCL cells results in pronounced CHOP induction (FIGS. 26A-26C) suggests that ER stress might play a role in NEO212-induced cell death.³⁰ This view is supported further by the observed decline in cyclin D protein levels (FIGS. 26A-26C). As it has been established⁴⁹ that ER stress results in down-regulation of this cell cycle regulator, our results are consistent with a role for ER stress. As well, NEO212 treatment resulted in the down-regulation of c-myc protein, which is noteworthy in view of this proto-oncoprotein's central role in cell proliferation and oncogenesis.³¹ Although not generally controlled by ER stress, there is an example in the literature where treatment of mouse or rat pre-adipocytes with palmitate resulted in aggravated ER stress (i.e., CHOP induction), along with down-regulation of c-myc (and cyclin D), followed by cellular apoptosis.⁵⁰ Altogether, these considerations point to the possibility that the ER stress-aggravating function of POH is preserved in the NEO212 molecule and possibly enhanced within the context of the chimeric construct.

In any case, NEO212-induced death of CTCL cells appears to be executed primarily via apoptosis. Proteolytic cleavage of caspases and PARP-1 protein, resulting in activation of caspases and inactivation of PARP-1, represents a well-established and widely-used marker of apoptotic cell death.^{51,52} In both HUT78 and HUT102 cells, NEO212 triggered the emergence of these markers at very low concentrations (0.1-1.0 μM). In MyLa cells, the same effect was observed, although higher (30 μM) NEO212 concentrations were required (FIGS. 25A-25D). Of note, the inclusion of commonly used antioxidants, i.e., ascorbic acid and beta-mercaptoethanol,^{34,35} largely prevented the induction of these apoptotic markers, suggesting that ROS might play a key role in mediating the cell death-inducing effects of NEO212. This model is consistent with our observation that direct addition of ROS to the cells, in the form of hydrogen peroxide, mimicked NEO212's effect on markers of apoptosis and proliferation (FIG. 27), and is further supported by earlier studies of NEO212 in human nasopharyngeal carcinoma and non-small cell lung cancer cells, which showed that NEO212 was able to trigger ROS accumulation in these cancer types in vitro.⁵³⁻⁵⁵ It therefore appears that NEO212's anticancer mechanism is at least in part similar to what has been reported for some of the well-established chemotherapeutic agents, such as doxorubicin and cisplatin, where the accumulation of ROS has been shown to further enhance their DNA-damaging and apoptosis-inducing potential.^{53,56}

In summary, we present data demonstrating the high anticancer potency of NEO212 in CTCL cell lines. Compared to both of its individual components, TMZ and POH, NEO212 exerts substantially greater cytotoxic activity, potentially via involvement of the pro-apoptotic module of the ER stress response mechanism, although its alkylating activity might contribute as well. As a next step in NEO212's development, in vivo experiments in immunodeficient murine CTCL models should be performed. We have attempted such models, but tumor take with our CTCL cell lines was unacceptably low, not inconsistent with reported challenges of achieving consistent tumor growth with these and other CTCL cell lines in general⁵⁷⁻⁵⁹ It is conceivable that serial passage of positive tumors in mice would yield more aggressive cells, and we are considering pursuing this approach. As well, we have performed toxicity studies in mice (see Supplemental Data in references^{16,17}) and Beagle dogs (unpublished) and determined that NEO212 is very well tolerated, which bodes well for future clinical studies.

### REFERENCES

1. Wilcox R A. Cutaneous T-cell lymphoma: 2016 update on diagnosis, risk-stratification, and management. Am J Hematol. 2016; 91(1):151-165.
2. McGirt L Y, Jia P, Baerenwald D A, et al. Whole-genome sequencing reveals oncogenic mutations in mycosis fungoides. Blood. 2015; 126(4):508-519.
3. Trautinger F, Eder J, Assaf C, et al. European Organisation for Research and Treatment of Cancer consensus recommendations for the treatment of mycosis fungoides/Sezary syndrome-Update 2017. Eur J Cancer. 2017; 77:57-74.
4. Jawed S I, Myskowski P L, Horwitz S, Moskowitz A, Querfeld C. Primary cutaneous T-cell lymphoma (mycosis fungoides and Sezary syndrome): part I. Diagnosis: clinical and histopathologic features and new molecular and biologic markers. J Am Acad Dermatol. 2014; 70(2):205 e201-216; quiz 221-202.
5. Korgavkar K, Xiong M, Weinstock M. Changing incidence trends of cutaneous T-cell lymphoma. JAMA Dermatol. 2013; 149(11):1295-1299.
6. Wilson L D, Hinds G A, Yu J B. Age, race, sex, stage, and incidence of cutaneous lymphoma. Clin Lymphoma Myeloma Leuk. 2012; 12(5):291-296.
7. Ringrose A, Zhou Y, Pang E, et al. Evidence for an oncogenic role of AHI-1 in Sezary syndrome, a leukemic variant of human cutaneous T-cell lymphomas. Leukemia. 2006; 20(9):1593-1601.
8. Willemze R. Mycosis fungoides variants-clinicopathologic features, differential diagnosis, and treatment. Semin Cutan Med Surg. 2018; 37(1):11-17.
9. Spicknall K E. Sezary syndrome-clinical and histopathologic features, differential diagnosis, and treatment. Semin Cutan Med Surg. 2018; 37(1):18-23.
10. Girardi M, Heald P W, Wilson L D. The pathogenesis of mycosis fungoides. N Engl J Med. 2004; 350(19):1978-1988.
11. Prince H M, Querfeld C. Integrating novel systemic therapies for the treatment of mycosis fungoides and Sezary syndrome. Best Pract Res Clin Haematol. 2018; 31(3):322-335.
12. Swerdlow S H, Campo E, Pileri S A, et al. The 2016 revision of the World Health Organization classification of lymphoid neoplasms. Blood. 2016; 127(20):2375-2390.
13. da Silva Almeida A C, Abate F, Khiabanian H, et al. The mutational landscape of cutaneous T cell lymphoma and Sezary syndrome. Nat Genet. 2015; 47(12):1465-1470.
14. Querfeld C, Zain J, Rosen S T. Primary Cutaneous T-Cell Lymphomas: Mycosis Fungoides and Sezary Syndrome. Cancer Treat Res. 2019; 176:225-248.
15. Larocca C, Kupper T. Mycosis Fungoides and Sezary Syndrome: An Update. Hematol Oncol Clin North Am. 2019; 33(1):103-120.
16. Chen T C, Cho H Y, Wang W, et al. A novel temozolomide-perillyl alcohol conjugate exhibits superior activity against breast cancer cells in vitro and intracranial triple-negative tumor growth in vivo. Mol Cancer Ther. 2014; 13(5):1181-1193.
17. Chen T C, Cho H Y, Wang W, et al. A novel temozolomide analog, NEO212, with enhanced activity against MGMT-positive melanoma in vitro and in vivo. Cancer Lett. 2015; 358(2):144-151.
18. Chen T C, Da Fonseca C O, Schönthal A H. Perillyl Alcohol and Its Drug-Conjugated Derivatives as Potential Novel Methods of Treating Brain Metastases. Int J Mol Sci. 2016; 17(9).
19. Jhaveri N, Agasse F, Armstrong D, et al. A novel drug conjugate targeting proneural and mesenchymal subtypes of patient-derived glioma cancer stem cells. Cancer Lett. 2015.
20. Crowell P L, Elson C E. Isoprenoids, Health and Disease. In: Wildman REC, ed. Neutraceuticals and Functional Foods. Boca Raton, Fla.: CRC Press; 2001.
21. Chen T C, Fonseca C O, Schönthal A H. Preclinical development and clinical use of perillyl alcohol for chemoprevention and cancer therapy. Am J Cancer Res. 2015; 5(5):1580-1593.
22. Chen T C, da Fonseca C O, Schonthal A H. Intranasal Perillyl Alcohol for Glioma Therapy: Molecular Mechanisms and Clinical Development. Int J Mol Sci. 2018; 19(12).
23. Chua J, Nafziger E, Leung D. Evidence-Based Practice: Temozolomide Beyond Glioblastoma. Curr Oncol Rep. 2019; 21(4):30.
24. Teimouri F, Nikfar S, Abdollahi M. Efficacy and side effects of dacarbazine in comparison with temozolomide in the treatment of malignant melanoma: a meta-analysis consisting of 1314 patients. Melanoma Res. 2013; 23(5):381-389.
25. Lee S Y. Temozolomide resistance in glioblastoma multiforme. Genes Dis. 2016; 3(3):198-210.
26. Roos W P, Kaina B. DNA damage-induced cell death: from specific DNA lesions to the DNA damage response and apoptosis. Cancer Lett. 2013; 332(2):237-248.
27. Kim S R, Lewis J M, Cyrenne B M, et al. BET inhibition in advanced cutaneous T cell lymphoma is synergistically potentiated by BCL2 inhibition or HDAC inhibition. Oncotarget. 2018; 9(49):29193-29207.
28. Harlow E, Lane D. Using Antibodies: A Laboratory Manual. Cold Spring Harbor, N.Y.: Cold Spring Harbor Laboratory; 1999.
29. Omura S, Asami Y, Crump A. Staurosporine: new lease of life for parent compound of today's novel and highly successful anti-cancer drugs. J Antibiot (Tokyo). 2018; 71(8):688-701.
30. Li Y, Guo Y, Tang J, Jiang J, Chen Z. New insights into the roles of CHOP-induced apoptosis in E R stress. Acta Biochim Biophys Sin (Shanghai). 2014; 46(8):629-640.
31. Carroll P A, Freie B W, Mathsyaraja H, Eisenman R N. The MYC transcription factor network: balancing metabolism, proliferation and oncogenesis. Front Med. 2018; 12(4):412-425.
32. Musgrove E A, Caldon C E, Barraclough J, Stone A, Sutherland R L. Cyclin D as a therapeutic target in cancer. Nat Rev Cancer. 2011; 11(8):558-572.
33. Srinivas U S, Tan B W Q, Vellayappan B A, Jeyasekharan A D. ROS and the DNA damage response in cancer. Redox Biol. 2018:101084.
34. Buettner G R. The pecking order of free radicals and antioxidants: lipid peroxidation, alpha-tocopherol, and ascorbate. Arch Biochem Biophys. 1993; 300(2):535-543.
35. Knight J A. Review: Free radicals, antioxidants, and the immune system. Ann Clin Lab Sci. 2000; 30(2):145-158.
36. Alpdogan O, Kartan S, Johnson W, Sokol K, Porcu P. Systemic therapy of cutaneous T-cell lymphoma (CTCL). Chin Clin Oncol. 2019; 8(1):10.
37. Zackheim H S. Topical carmustine (BCNU) in the treatment of mycosis fungoides. Dermatol Ther. 2003; 16(4):299-302.
38. Tani M, Fina M, Alinari L, Stefoni V, Baccarani M, Zinzani P L. Phase II trial of temozolomide in patients with pretreated cutaneous T-cell lymphoma. Haematologica. 2005; 90(9):1283-1284.
39. Querfeld C, Rosen S T, Guitart J, et al. Multicenter phase II trial of temozolomide in mycosis fungoides/sezary syndrome: correlation with O(6)-methylguanine-DNA methyltransferase and mismatch repair proteins. Clin Cancer Res. 2011; 17(17):5748-5754.
40. Bird T G, Whittaker S, Wain E M, Child F, Morris S L. Temozolomide for central nervous system involvement in mycosis fungoides. Int J Dermatol. 2016; 55(7):751-756.
41. Brada M, Judson I, Beale P, et al. Phase I dose-escalation and pharmacokinetic study of temozolomide (SCH 52365) for refractory or relapsing malignancies. Br J Cancer. 1999; 81(6):1022-1030.
42. Hammond L A, Eckardt J R, Baker S D, et al. Phase I and pharmacokinetic study of temozolomide on a daily-for-5-days schedule in patients with advanced solid malignancies. J Clin Oncol. 1999; 17(8):2604-2613.
43. Chamberlain M C. Temozolomide: therapeutic limitations in the treatment of adult high-grade gliomas. Expert Rev Neurother. 2010; 10(10):1537-1544.
44. Hegi M E, Diserens A C, Gorlia T, et al. MGMT gene silencing and benefit from temozolomide in glioblastoma. N Engl J Med. 2005; 352(10):997-1003.
45. Kaina B, Christmann M. DNA repair in personalized brain cancer therapy with temozolomide and nitrosoureas. DNA Repair (Amst). 2019; 78:128-141.
46. Cho H Y, Wang W, Jhaveri N, et al. Perillyl alcohol for the treatment of temozolomide-resistant gliomas. Mol Cancer Ther. 2012; 11(11):2462-2472.
47. Knizhnik A V, Roos W P, Nikolova T, et al. Survival and death strategies in glioma cells: autophagy, senescence and apoptosis triggered by a single type of temozolomide-induced DNA damage. PLoS One. 2013; 8(1):e55665.
48. Schonthal A H. Pharmacological targeting of endoplasmic reticulum stress signaling in cancer. Biochem Pharmacol. 2013; 85(5):653-666.
49. Brewer J W, Hendershot L M, Sherr C J, Diehl J A. Mammalian unfolded protein response inhibits cyclin D1 translation and cell-cycle progression. Proc Natl Acad Sci USA. 1999; 96(15):8505-8510.
50. Guo W, Wong S, Xie W, Lei T, Luo Z. Palmitate modulates intracellular signaling, induces endoplasmic reticulum stress, and causes apoptosis in mouse 3T3-L1 and rat primary preadipocytes. Am J Physiol Endocrinol Metab. 2007; 293(2):E576-586.
51. Soldani C, Scovassi A I. Poly(ADP-ribose) polymerase-1 cleavage during apoptosis: an update. Apoptosis. 2002; 7(4):321-328.
52. Li J, Yuan J. Caspases in apoptosis and beyond. Oncogene. 2008; 27(48):6194-6206.
53. Marullo R, Werner E, Degtyareva N, et al. Cisplatin induces a mitochondrial-ROS response that contributes to cytotoxicity depending on mitochondrial redox status and bioenergetic functions. PLoS One. 2013; 8(11):e81162.
54. Song X, Xie L, Wang X, et al. Temozolomide-perillyl alcohol conjugate induced reactive oxygen species accumulation contributes to its cytotoxicity against non-small cell lung cancer. Sci Rep. 2016; 6:22762.
55. Xie L, Song X, Guo W, et al. Therapeutic effect of TMZ-POH on human nasopharyngeal carcinoma depends on reactive oxygen species accumulation. Oncotarget. 2016; 7(2):1651-1662.
56. Conklin K A. Chemotherapy-associated oxidative stress: impact on chemotherapeutic effectiveness. Integr Cancer Ther. 2004; 3(4):294-300.
57. Netchiporouk E, Gantchev J, Tsang M, et al. Analysis of CTCL cell lines reveals important differences between mycosis fungoides/Sezary syndrome vs. HTLV-1(+) leukemic cell lines. Oncotarget. 2017; 8(56):95981-95998.
58. Doebbeling U. A mouse model for the Sezary syndrome. J Exp Clin Cancer Res. 2010; 29:11.
59. Thaler S, Burger A M, Schulz T, et al. Establishment of a mouse xenograft model for mycosis fungoides. Exp Dermatol. 2004; 13(7):406-412.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

What is claimed is:

1. A method for treating a primary cutaneous lymphoma mycosis fungoides in a mammal, the method comprising administering to the mammal a therapeutically effective amount of a perillyl alcohol carbamate.

2. The method of claim 1, wherein the primary cutaneous lymphoma is a cutaneous T cell lymphoma (CTCL).

3. The method of claim 2, wherein the cutaneous T cell lymphoma (CTCL) is mycosis fungoides, primary cutaneous anaplastic large cell lymphoma (ALCL), or Sezary syndrome.

4. The method of claim 2, wherein the cutaneous T cell lymphoma (CTCL) is mycosis fungoides.

5. The method of claim 1, wherein the perillyl alcohol carbamate is perillyl alcohol conjugated with a therapeutic agent.

6. The method of claim 5, wherein the therapeutic agent is a chemotherapeutic agent.

7. The method of claim 6, wherein the chemotherapeutic agent is selected from the group consisting of a DNA alkylating agent, a topoisomerase inhibitor, an endoplasmic reticulum stress inducing agent, a platinum compound, an antimetabolite, an enzyme inhibitor, and a receptor antagonist.

8. The method of claim 5, wherein the therapeutic agent is selected from the group consisting of dimethyl celecoxib (DMC), temozolomide (TMZ) and rolipram.

9. The method of claim 1, wherein the perillyl alcohol carbamate is selected from the group consisting of (a) 4-(bis-N,N′-4-isopropenyl cyclohex-1-enylmethyloxy carbonyl [5-(2,5-dimethyl phenyl)-3-trifluoromethyl pyrazol-1-yl] benzenesulfonamide; (b) 4-(3-cyclopentyloxy-4-methoxy phenyl)-2-oxo-pyrrolidine-1-carboxylic acid 4-isopropenyl cyclohex-1-enylmethyl ester; and (c) 3-methyl 4-oxo-3,4-dihydroimidazo[5,1-d][1,2,3,5]tetrazine-8-carbonyl)-carbamic acid-4-isopropenyl cyclohex-1-enylmethyl ester.

10. The method of claim 1, further comprising treating the mammal with radiation.

11. The method of claim 1, further comprising administering to the mammal a chemotherapeutic agent.

12. The method of claim 1, wherein the perillyl alcohol carbamate is administered by inhalation, intranasally, orally, intravenously, subcutaneously or intramuscularly.

13. The method of claim 1, wherein the mammal is a human.
